(19) United States
(12) Patent Application Publication
(10) Pub. No.: US 2008/0009628 A1
(43) Pub. Date: Jan. 10, 2008

(54) One-Pot Condensation-Reduction Methods for Preparing Substituted Allylic Alcohols

(76) Inventors: Michael Reuman, New Hope, PA (US); Roger Faessler, Stetten (CH); Armin Roessler, Tengen (DE); Kirk Sorgi, Doylestown, PA (US); Xun Li, New Hope, PA (US); Jeffrey S. Grimm, Branchburg, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/762,371

(22) Filed: Jun. 13, 2007

Related U.S. Application Data
(60) Provisional application No. 60/818,551, filed on Jul. 5, 2006.

(51) Int. Cl. C07D 401/02; C07D 209/48; C07D 211/06; C07D 211/96

(52) U.S. Cl. 546/156; 546/152; 546/200; 546/245; 546/248; 548/477

(57) ABSTRACT

One-pot condensation-reduction methods for preparing substituted allylic alcohols as well as highly selective extractive methods to separate isomeric alcohols produced in the one-pot condensation-reduction processes are provided for preparing, for example, a quinolone.

### CROSS REFERENCE TO RELATED APPLICATIONS

This present application claims benefit of U.S. Provisional Patent Application Ser. No. 60/818,551, filed Jul. 5, 2006, which is incorporated herein by reference in its entirety and for all purposes.

### FIELD OF THE INVENTION

The invention is related to one-pot methods for the production of substituted allylic alcohols as well as extractive methods for the separation of certain isomeric alcohol products from such one-pot methods, which are useful for preparing, for example, a quinolone.

### BACKGROUND OF THE INVENTION

PCT PUB WO 2005/033108A1 describes the preparation of fluorovinylallylic alcohols, chlorovinylallylic alcohols and related intermediates and their use in the preparation of 7-amino alkylidenyl-heterocyclic quinolone and naphthyridones. These compounds are novel antimicrobial agents.

J. Org. Chem. 58, 5683 (1993), Bioorganic & Medicinal Chemistry 10, 929 (2002), Bioorganic & Medicinal Chemistry 11, 2403 (2003) and other publications describe the preparation of fluorovinyl and related allylic alcohols by classical, discrete two-step methods. The first step is a Horner-Emmons coupling reaction with a phosphonate derivative such as triethyl-2-fluoro-2-phosphonoacetate and a ketone or aldehyde to give an unsaturated ester. The ester is then isolated before being subjected to reduction with reagents such as diisobutyl aluminum hydride (DIBAL) or lithium aluminum hydride (LAH) to give the allylic alcohol. The resulting isomeric alcohols are separated into individual isomers by column chromatography.

All documents cited herein are incorporated by reference.

### SUMMARY OF THE INVENTION

The invention provides a method for making one or more compounds of Formula (1),

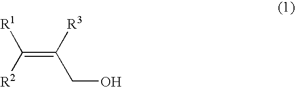

wherein
R¹ and R² are independently selected from H, aryl, C₁₋₁₀alkyl, C₂₋₁₀alkenyl, and C₃₋₁₀alkynyl, or R¹, R² and the C atom they attach to may together form C₃₋₁₀cycloalkyl or heterocyclyl; and
R³ is H, C₁₋₁₀alkyl, C₂₋₁₀alkenyl, C₃₋₁₀alkynyl, halogen, aryl, heteroaryl, or heterocyclyl,

said method comprising

(a) reacting, in the presence of one or more bases, one or more compounds of Formula (i)

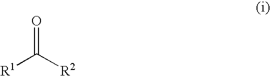

wherein R¹ and R² are independently selected from H, aryl, C₁₋₁₀alkyl, C₂₋₁₀alkenyl, and C₃₋₁₀alkynyl, or R¹, R² and the C atom they attach to may together form C₃₋₁₀cycloalkyl or heterocyclyl,

with a compound of Formula (ii)

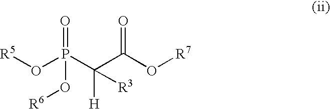

wherein R³ is selected from H, C₁₋₁₀alkyl, C₂₋₁₀alkenyl, C₃₋₁₀alkynyl, halogen, aryl, heteroaryl, and heterocyclyl, and R⁵, R⁶, and R⁷ are independently selected from C₁₋₁₀alkyl and aryl; and

(b) adding one or more reducing agents into the reaction of step (a).

The present invention also provides a method for separating isomeric alcohols of Formula (1) in an aqueous mixture

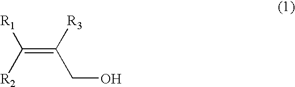

wherein
R¹ and R² are different groups selected from H, C₁₋₁₀alkyl, C₂₋₁₀alkenyl, and C₃₋₁₀alkynyl, or R¹, R² and the C atom they attach to may together form an asymmetric group selected from substituted C₃₋₁₀cycloalkyl and optionally substituted heterocyclyl;
R³ is H, unsubstituted C₁₋₁₀alkyl, halogen, aryl, or heterocyclyl,

said method comprising
(a) contacting an aqueous mixture of the isomeric alcohols with an adequate volume of a non-polar solvent; and
(b) separating the resulting non-polar solvent from the aqueous layer.

The present invention further provides a method for separating isomers of Formula (2) in an n-butanol solution

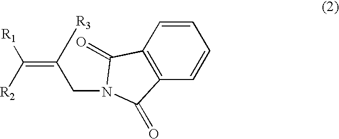

wherein
R¹, R² and the C atom they attach to together form

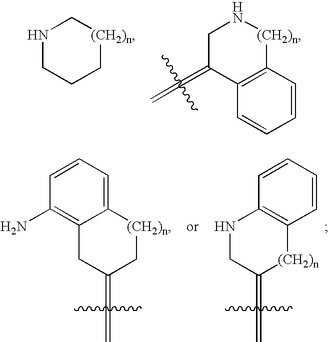

R³ is H, unsubstituted C₁₋₁₀alkyl, halogen, aryl, or heterocyclyl; and
n is 0-4,

said method comprising
(a) contacting an aqueous mixture of the isomers of Formula (2) with an adequate volume of a mixture of HCl and IPA;
(b) heating the resulting solution to a temperature from about 85° C. to about 118° C.; and
(c) adding IPA into the resulting solution.

### DETAILED DESCRIPTION OF THE INVENTION

The present invention is directed to one-pot methods for the production of substituted allylic alcohols, which eliminates all isolation, extraction, and/or concentration step(s) before the reduction step that follows. In particular, the present invention is directed to a more scalable, non-chromatographic process for making various quantities of, including large quantity production such as on the scale of kilogram (Kg or kg), of substituted allylic alcohols. One advantage of eliminating all isolation, extraction, and/or concentration step(s), usually performed after the Horner-Wadsworth-Emmons or alternate coupling reaction step, is the minimization of decomposition of the intermediate unsaturated esters that may occur with the classical, discrete two-step methods. Additionally, the one-pot methods of the present invention are easier to carry out and provide savings of various reagents as well as time.

Specifically, the present invention provides a method for making one or more compounds of Formula (1),

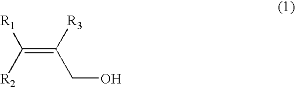

wherein
R¹ and R² are independently selected from H, aryl, C₁₋₁₀alkyl, C₂₋₁₀alkenyl, and C₃₋₁₀alkynyl, or R¹, R² and the C atom they attach to may together form C₃₋₁₀cycloalkyl or heterocyclyl; and
R³ is H, C₁₋₁₀alkyl, C₂₋₁₀alkenyl, C₃₋₁₀alkynyl, halogen, aryl, heteroaryl, or heterocyclyl,

said method comprising
(a) reacting, in the presence of one or more bases, one or more compounds of Formula (i)

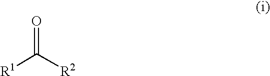

wherein R¹ and R² are independently selected from H, aryl, C₁₋₁₀alkyl, C₂₋₁₀alkenyl, and C₃₋₁₀alkynyl, or R¹, R² and the C atom they attach to may together form C₃₋₁₀cycloalkyl or heterocyclyl,

with a compound of Formula (ii)

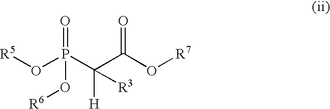

wherein R³ is selected from H, C₁₋₁₀alkyl, C₂₋₁₀alkenyl, C₃₋₁₀alkynyl, halogen, aryl, heteroaryl, and heterocyclyl, and R⁵, R⁶, and R⁷ are independently selected from C₁₋₁₀alkyl and aryl; and
(b) adding one or more reducing agents into the reaction of step (a).

In particular, the compound of Formula (1) is

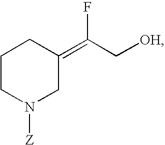

wherein Z is selected from —C(O)O—C(CH₃)₃, —C(O)OCH₂Ph, —C(O)-Ph, —C(O)CH₃, —S(O)₂-PhCH₃, and —S(O)₂—CH₃. More particularly, the compound of Formula (1) is

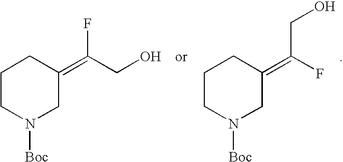

More particularly, the compound of Formula (1) consists of isomeric alcohols

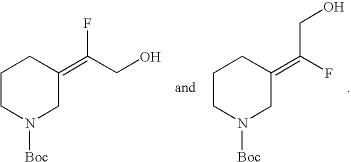

In particular, the compound of Formula (i) is

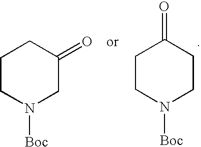

Particularly, the compound of Formula (i) is in one or more solvents independently selected from alcohol, 2-methoxyethanol, diols, polyols, polyethers, polyethylene glycol monomethyl ether derivatives, TFA, DMA, DMF, pyridine, and Et₃N. More particularly, the solvent is one or more alcohols, each alcohol having 1-6 carbon atoms. More particularly, the solvent is 2-methoxyethanol or ethanol. Alternatively, the compound of Formula (i) can also be in one or more solvents independently selected from THF, Et₂O, n-butanol, and toluene.

In particular, the base is at least one member selected from metal carbonates, bicarbonates, metal hydroxides, and organic bases. More particularly, the base is at least one member selected from Cs₂CO₃, K₂CO₃, KOt-Bu, Li₂CO₃, Na₂CO₃, LiOH, NaOH, KOH, Et₃N, DBU, DABCO, and pyridine. More particularly, the base is Cs₂CO₃.

In particular, the reducing agent is one or more metal borohydrides. More particularly, the reducing agent is at least one member selected from NaBH₄, LiBH₄, KBH₄, Ca(BH₄)₂, and Zn(BH₄)₂. In addition, when the reducing agent is one or more metal borohydrides, it is contemplated that one or more salts compatible with such metal borohydride(s) can be added. The introduction of such compatible salts can lead to reagent's different reactivity profile in the reduction step, but it will not adversely affect the reducing function of the reducing agent(s). Thus, according to the present invention, the method for making one or more compounds of Formula (1) further comprises adding a compatible salt in step (b). For example, when the reducing agent is NaBH₄, the compatible salt can be LiCl or CaCl₂ or both. Alternatively, when the compound of Formula (i) is in polyethers, Et₃N, THF, Et₂O, or toluene, the reducing agent is at least one member selected from DIBAL and LAH.

More particularly,

the compound of Formula (1) is

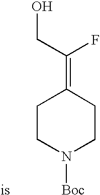

or a mixture of

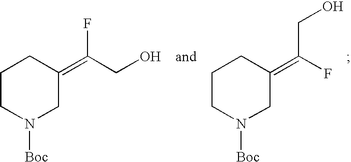

the compound of Formula (i) is selected from

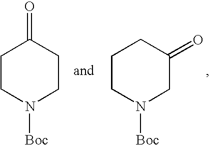

said compound of Formula (i) is in the solvent of 2-methoxyethanol;
the base is Cs₂CO₃; and
the reducing agent is NaBH₄.

More particularly,
the compounds of Formula (1) are

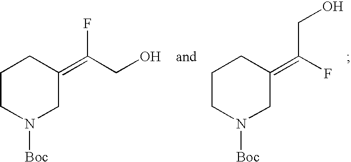

the compound of Formula (i) is

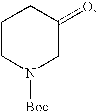

said compound of Formula (i) is in the solvent of 2-methoxyethanol;
the base is Cs₂CO₃; and
the reducing agent is NaBH₄.

According to the present invention, one example of the method for making a compound of Formula (1) comprises
(a) reacting

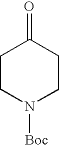

in the solvent of 2-methoxyethanol
   with Cs₂CO₃ and

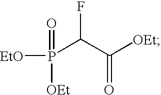

and
(b) adding NaBH₄ into the reaction of step (a) to form

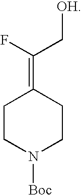

Another example of the invention is the one-pot coupling-reduction sequence

from

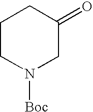

using Cs₂CO₃ and

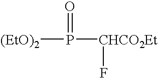

followed by NaBH₄ to prepare

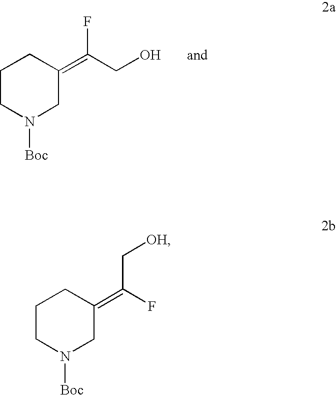

which are referred to herein as E-isomer and Z-isomer, respectively. The E:Z ratio in such preparation can vary; for example, it can be about 1:1 (±20%). More particularly, according to the present invention, the method for making one or more compounds of Formula (1) comprises

(a) reacting

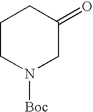

in the solvent of 2-methoxyethanol with Cs₂CO₃ and

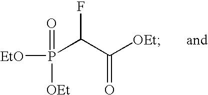

(b) adding NaBH₄ into the reaction of step (a) to form a mixture of

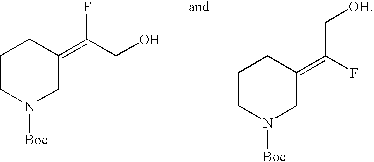

Particularly, both steps (a) and (b) of the method according to the present invention are done in one reaction vessel. More particularly, according to the present invention, the method for making one or more compounds of Formula (1) further comprises (c) a liquid-liquid extraction with a two-phase mixture composed of a polar and a non-polar phase after step (b).

In addition, the present invention is also directed to novel extractive methods for the separation of isomers of certain alcohols produced by the one-pot methods described herein. The novel extractive methods eliminate the need for a chromatography step to separate certain isomeric alcohols produced by the one-pot methods of the present invention.

Specifically, the present invention also provides a method for separating isomeric alcohols of Formula (1) in an aqueous mixture

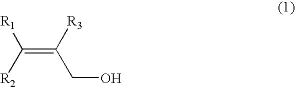

wherein
R¹ and R² are different groups selected from H, C₁₋₁₀alkyl, C₂₋₁₀alkenyl, and C₂₋₁₀alkynyl, or R¹, R² and the C atom they attach to may together form an asymmetric group selected from substituted C₃₋₁₀cycloalkyl and optionally substituted heterocyclyl;
R³ is H, unsubstituted C₁₋₁₀alkyl, halogen, aryl, or heterocyclyl,
   said method comprising
(a) contacting an aqueous mixture of the isomeric alcohols with an adequate volume of a non-polar solvent; and
(b) separating the resulting non-polar solvent from the aqueous layer.

Particularly, R¹, R² and the C atom they attach to together form an asymmetric group selected from

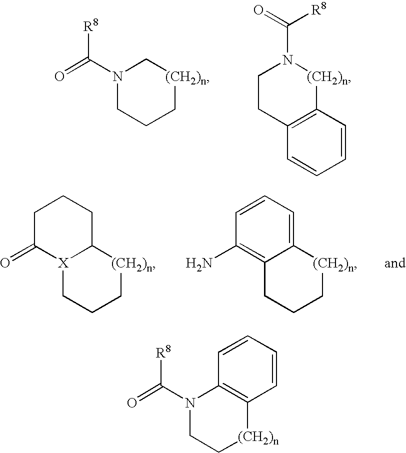

wherein
n is 0-4;
X is N or CH; and
R⁸ is C₁₋₁₀alkyl, C₁₋₁₀alkoxy, aryloxy, or aryl,
   provided that the C atom R¹ and R² attach to is not next to a N atom in the asymmetric group.

More particularly, the asymmetric group is selected from

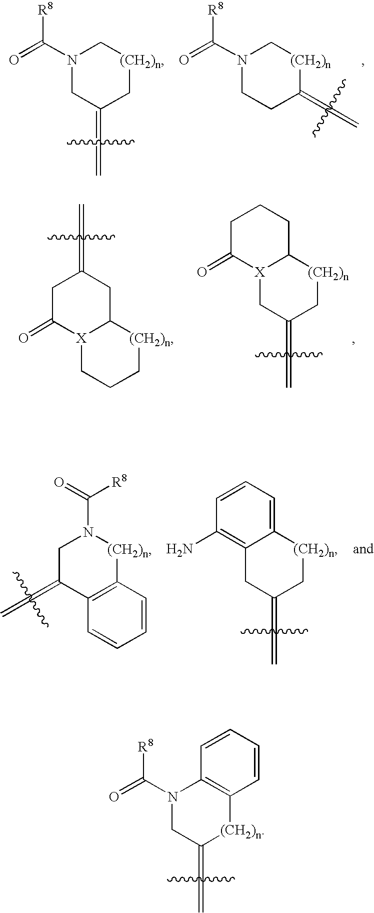

More particularly, the asymmetric group is selected from

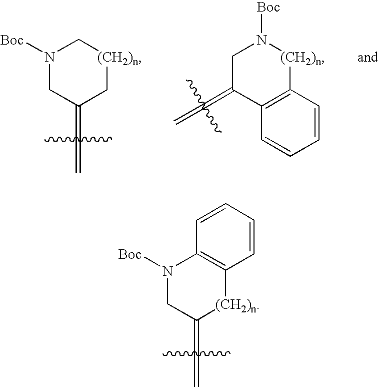

More particularly, the asymmetric group is

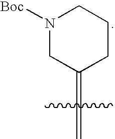

In addition, the extractive methods of the present invention further comprise (c) contacting the aqueous layer with an adequate volume of a water-insoluble polar solvent. In particular, the water-insoluble polar solvent is methyl tert-butyl ether or ethyl acetate.

Particularly, the non-polar solvent is hexane or heptane. More particularly, the non-polar solvent is hexane or heptane and the polar solvent is methyl tert-butyl ether.

For example, in the present methods for the preparation of substituted allylic alcohols, such as the alcohol 2′

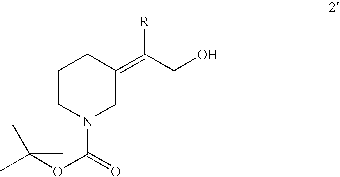

wherein R is H, alkyl, halogen, aryl, or heterocyclyl, the extraction process can be modified. The aqueous product mixture can be extracted with a non-polar hydrocarbon solvent, preferably heptane, to provide the less polar isomer after removal of this solvent. Next the aqueous layer is extracted with a more polar solvent, such as methyl tert-butyl ether. This solution is concentrated to provide the more polar isomer.

A preferred process, as illustrated in Scheme 1,

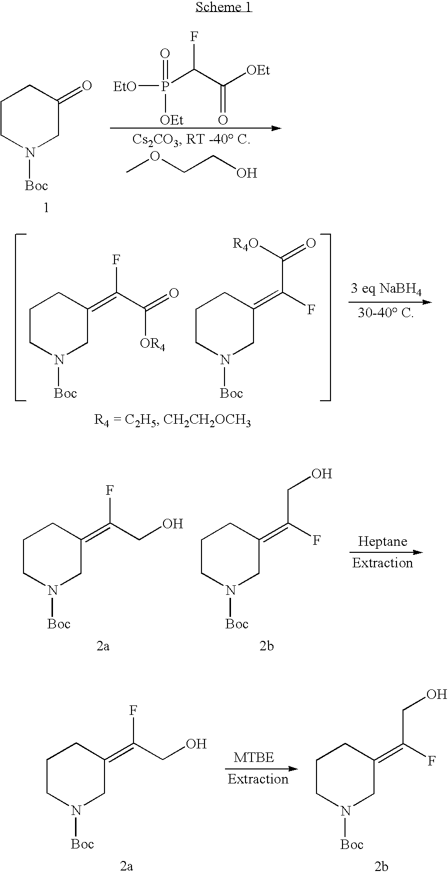

shows the preparation of alcohol 2

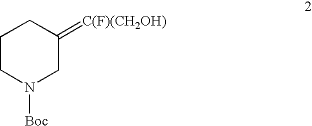

and the extractive separation into highly enriched components 2a and 2b. As noted above, this separation previously would be done by less convenient methods such as column chromatography. The extractive method for separation of isomeric alcohols is part of the new process in this invention. The extractive efficiency may vary according to the structures of the molecules involved, such as those of 2, in which the alcohol group of one isomer is in close proximity to a polar group or hydrogen bond accepting group.

This selective extraction process of the present invention, which relates to the one-pot coupling-reduction method using Cs₂CO₃ followed by NaBH₄, eliminates the need for any chromatography to separate isomeric alcohols at this stage. The selectivity in this process can, in part, be related to the proximity of the alcohol OH group and the Boc carbonyl. For instance, in the case of alcohol 2, the E-isomer molecular modeling places these groups about 2 Å apart; however in the Z-isomer, the distance is greater than 3 Å, which indicates that in the E-isomer the OH group can form an intramolecular hydrogen bond with the Boc carbonyl group. This possible attribute, among others, can make the E-isomer more readily extracted into a non-polar solvent than the Z-isomer.

The present invention also provides a method for separating isomers of Formula (2) in an n-butanol solution

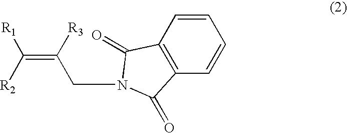

wherein
R¹, R² and the C atom they attach to together form

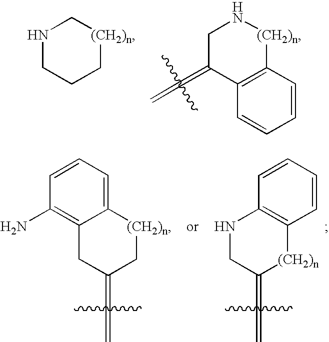

R³ is H, unsubstituted C₁₋₁₀alkyl, halogen, aryl, or heterocyclyl; and
n is 0-4,

said method comprising
(a) contacting an aqueous mixture of the isomers of Formula (2) with an adequate volume of a mixture of HCl and IPA;
(b) heating the resulting solution to a temperature from about 85° C. to about 118° C.; and
(c) adding IPA into the resulting solution.

In particular, the mixture of HCl and IPA is 5-6N HCl in 2-propanol. More particularly, vacuum is applied in step (b) (heating the resulting solution up to about 110° C.). More particularly, the solution in step (b) is heated to about 110° C. In addition, the method for separating isomers of Formula (2) further comprises (d) cooling the resulting solution to a temperature between r.t. and −20° C. More particularly, the temperature in step (d) is between −15 and −20° C.

Particularly, the isomers of Formula (2) are

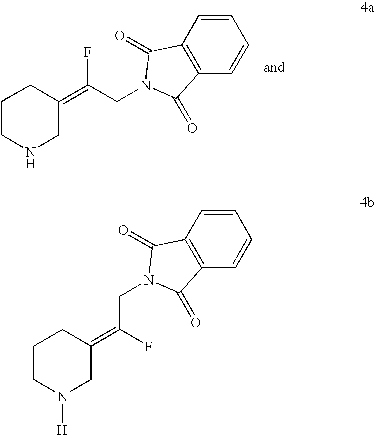

in n-butanol; the mixture of HCl and IPA is 5-6N HCl in 2-propanol; and the solution in step (b) is heated to about 110° C. under vacuum.

One such example of the invention, which also relates to the one-pot coupling-reduction method using Cs₂CO₃ followed by NaBH₄, is a selective crystallization process that eliminates the need for any chromatography to separate isomers such as

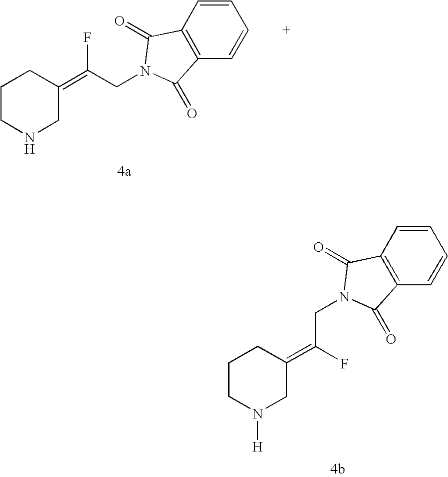

during the process, as shown in Scheme 2 blow:

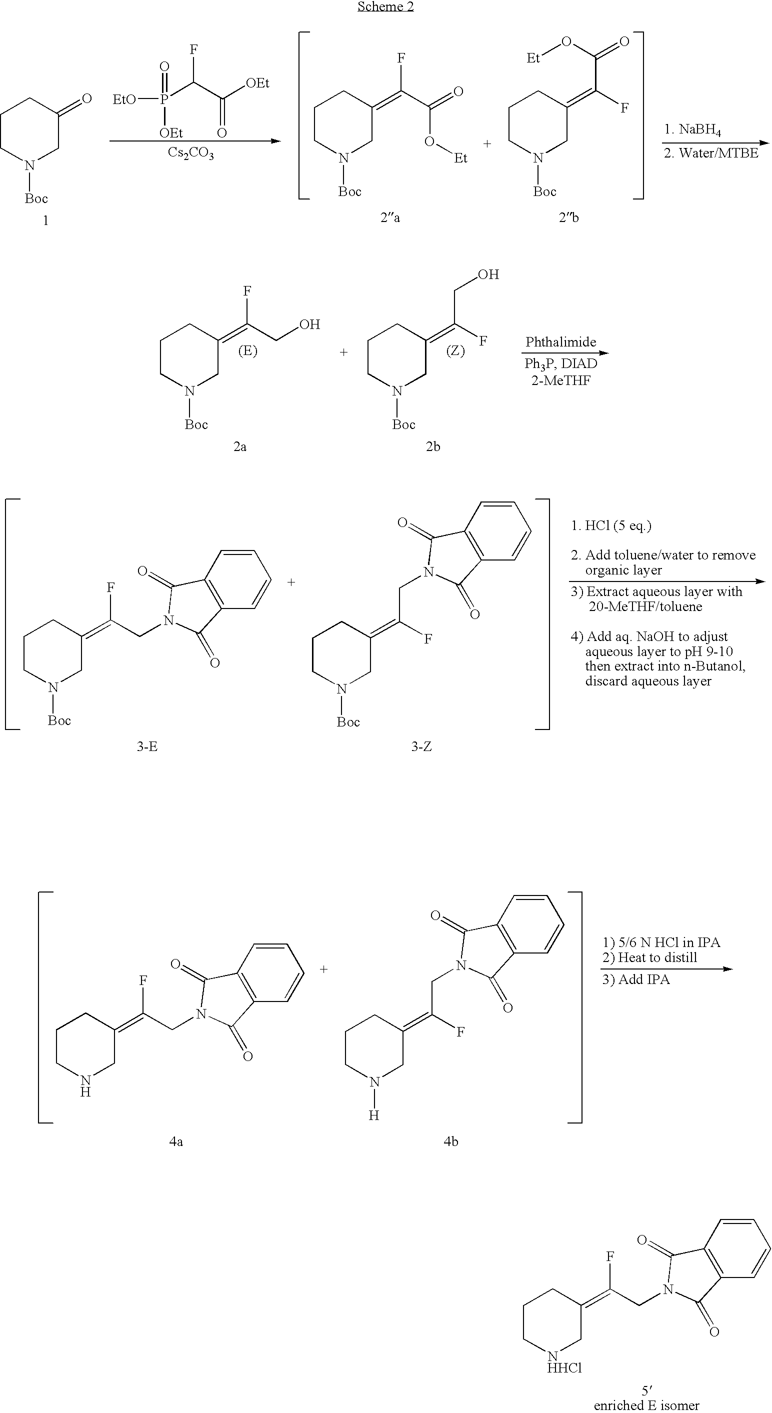

Also included in the present invention is synthesis of a compound useful as a topoisomerase inhibitor having the structure below:

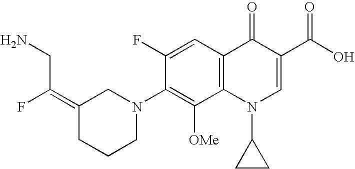

said method comprising
(a) reacting

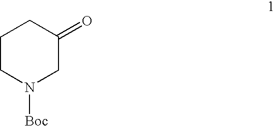

in the presence of one or more bases

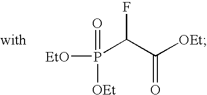

(b) adding one or more reducing agents into the reaction of step (a) to form

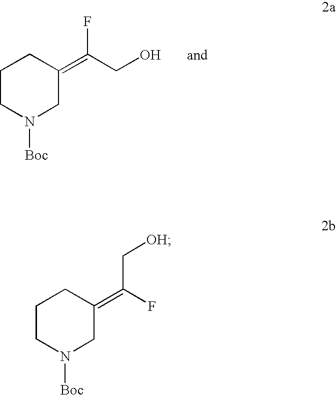

(c) extracting

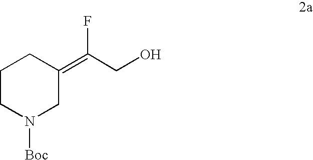

with hexane or heptane;
(d) converting

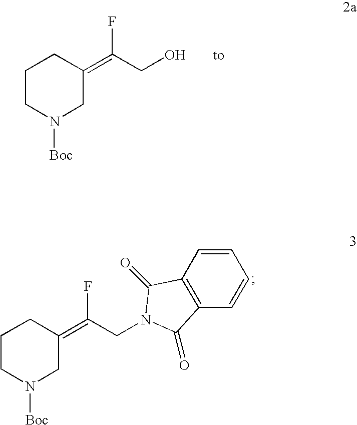

(e) converting

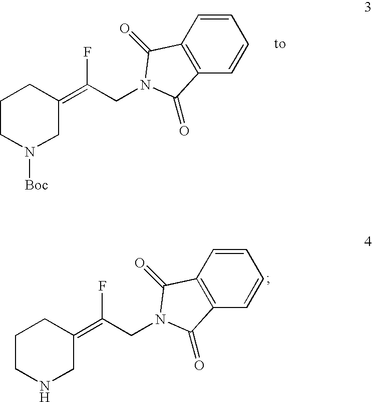

(f) reacting

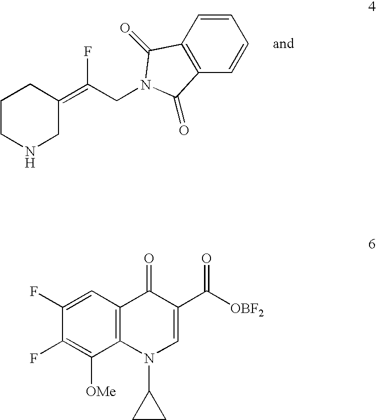

to form

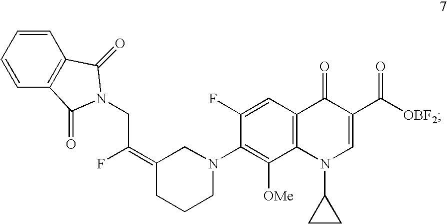

(g) converting

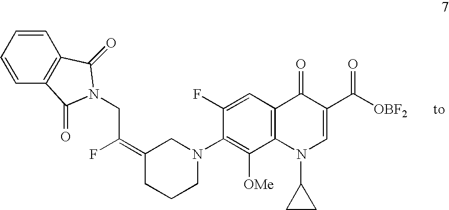

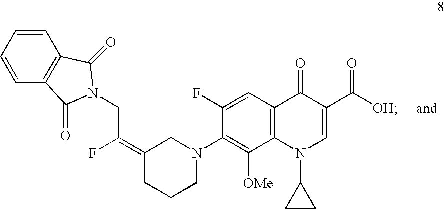

adding H₂NNH₂ into

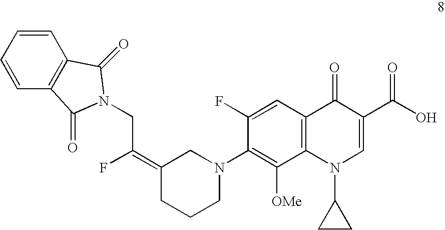

and MeOH.

In one example of this method of the invention,

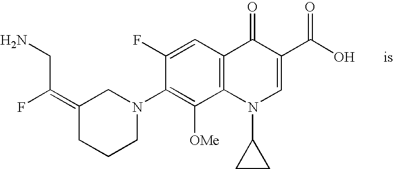

further converted into

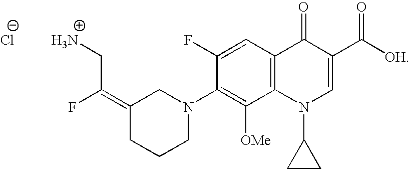

Further included in the present invention is synthesis of a compound useful as a topoisomerase inhibitor having the structure below:

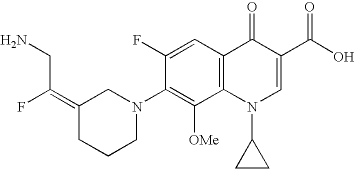

said method comprising
(a) reacting

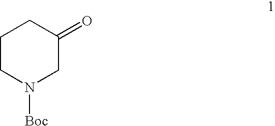

in the presence of one or more bases

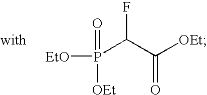

(b) adding one or more reducing agents into the reaction of step (a) to form

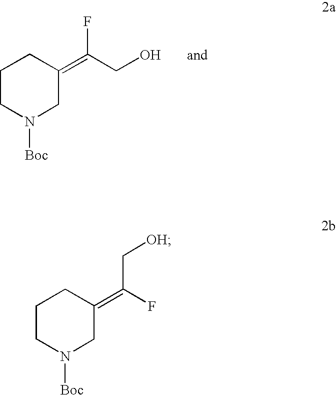

(c) extracting

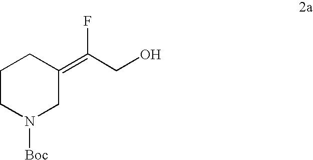

with hexane or heptane;
(d) converting

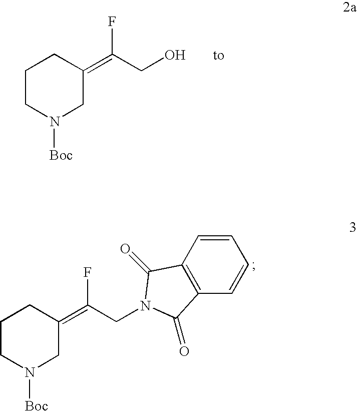

(e) converting

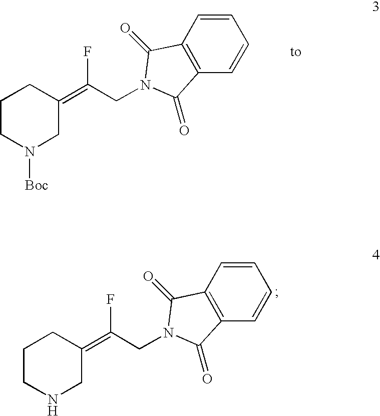

(f) reacting

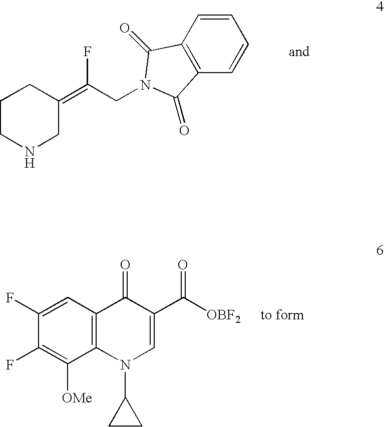

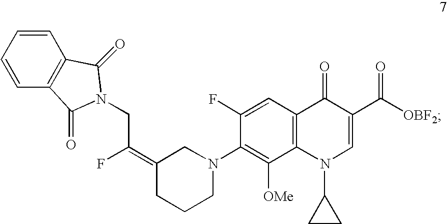

(g) converting

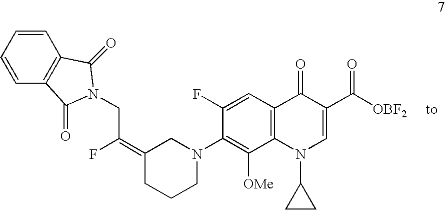

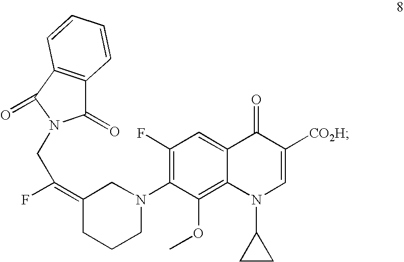

(h) converting

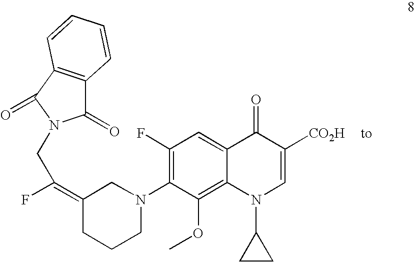

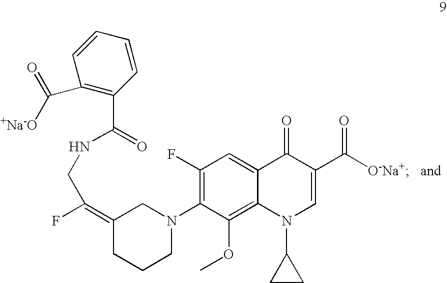

(i) adding MeOH and H₂SO₄, sequentially, into the reaction of step (h).

Another example of the present invention is synthesis of a compound useful as a topoisomerase inhibitor having the structure below:

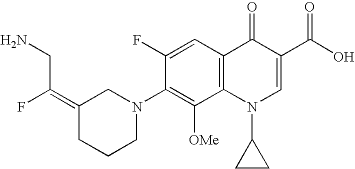

said method comprising
(a) reacting

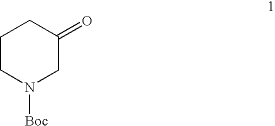

in the presence of one or more bases

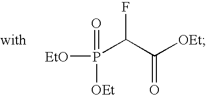

(b) adding one or more reducing agents into the reaction of step (a) to form

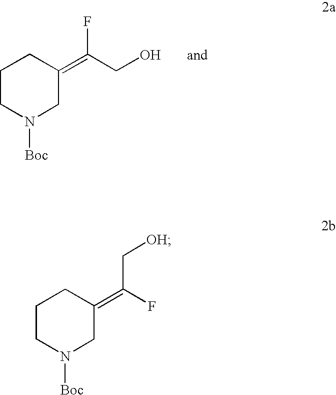

(c) converting

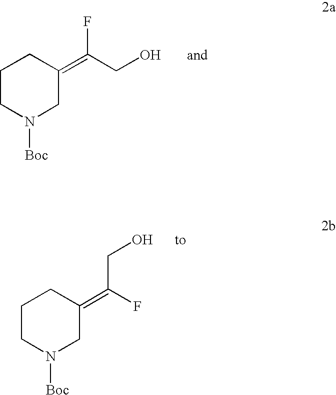

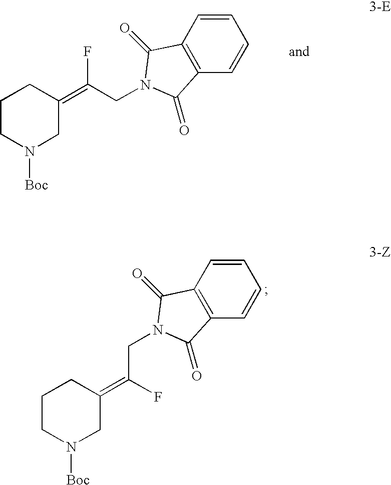

(d) converting

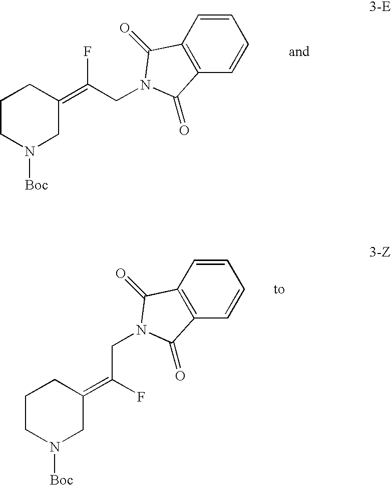

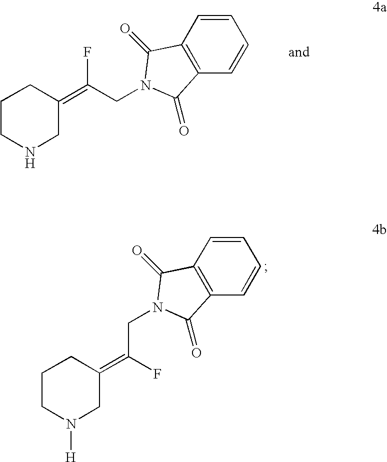

(e) adding 5-6 N HCl in IPA into the reaction of step (d);
(f) heating the reaction of step (e) to about 110° C.;
(g) adding IPA to precipitate

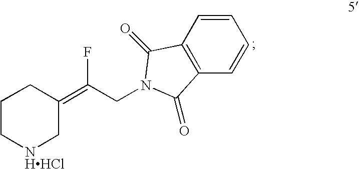

(h) converting

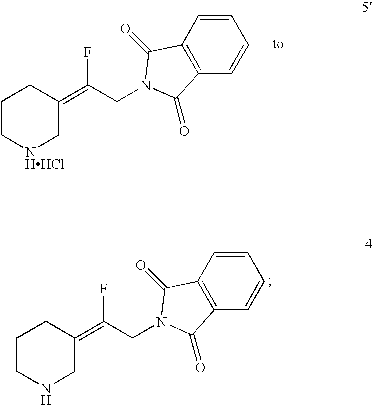

(i) reacting

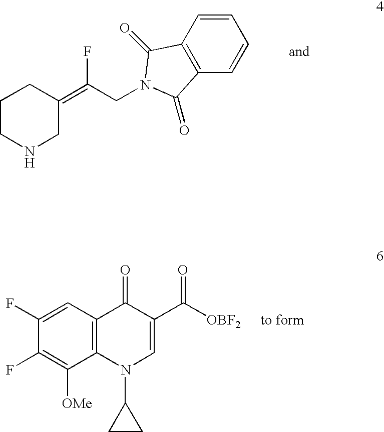

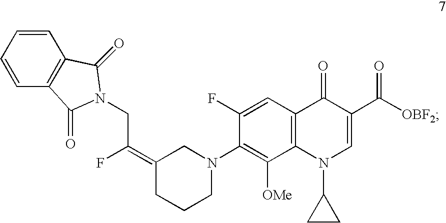

(j) converting

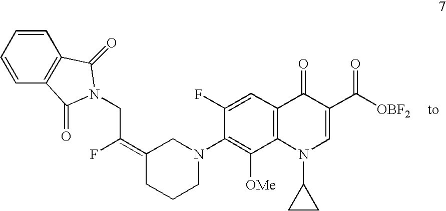

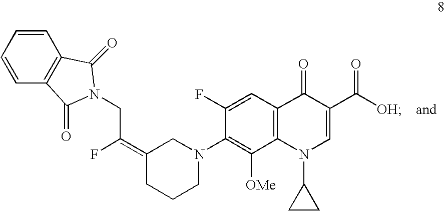

(k) adding H₂NNH₂ into

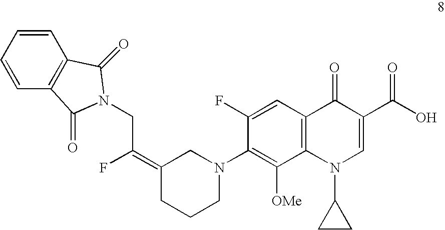

and MeOH.

In one such example of this method of the invention, one or more extractions using one or more solvents selected from alcohol and non-polar aprotic can be performed in step (d). Particularly, the solvent is selected from 2-propanol, 2-MeTHF, toluene, diethyl ether, ethyl acetate, MTBE, and n-butanol. More particularly, the solvents are 2-MeTHF and toluene. More particularly, the solvent is n-butanol. More particularly, one extraction with 2-MeTHF and toluene is performed followed by another extraction with n-butanol.

Yet another example of the present invention is synthesis of a compound useful as a topoisomerase inhibitor having the structure below:

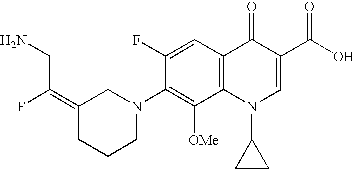

said method comprising
(a) reacting

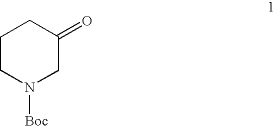

in the presence of one or more bases

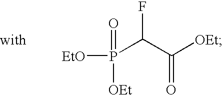

(b) adding one or more reducing agents into the reaction of step (a) to form

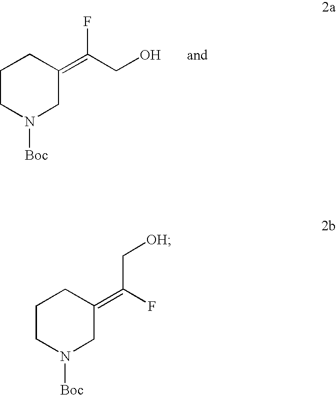

(c) converting

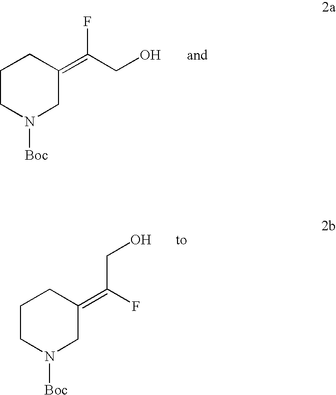

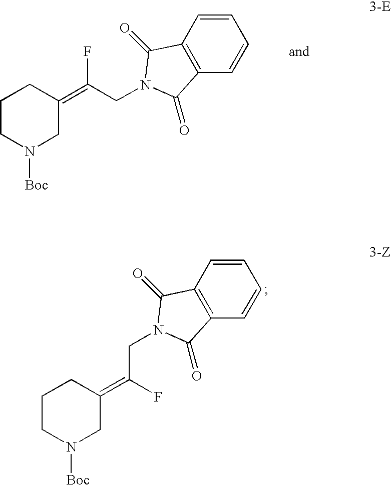

(d) converting

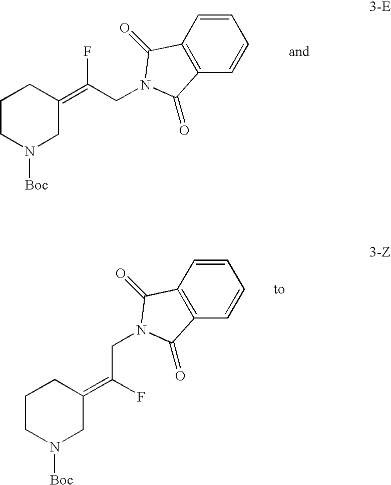

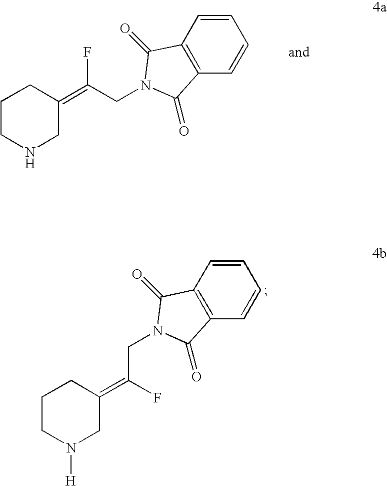

(e) adding 5-6 N HCl in IPA into the reaction of step (d);
(f) heating the reaction of step (e) to about 110° C.;
(g) adding IPA to precipitate

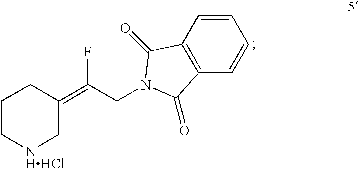

(h) converting

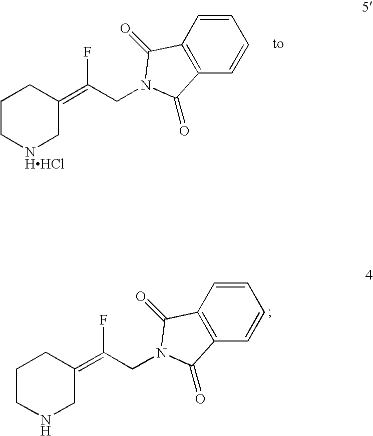

(i) reacting

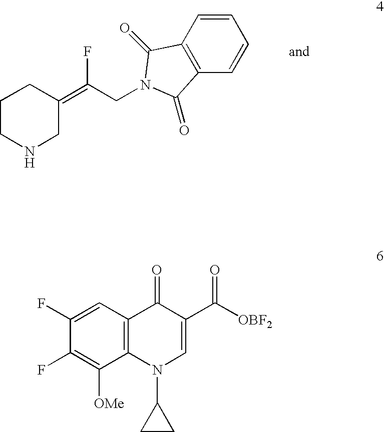

to form

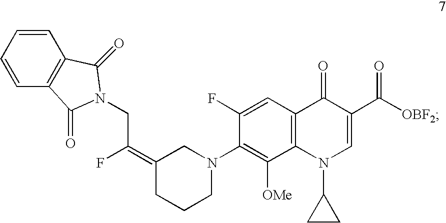

(j) converting

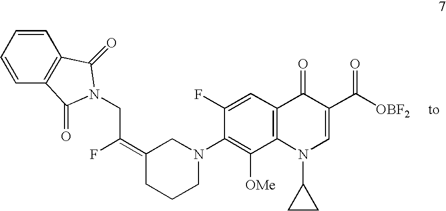

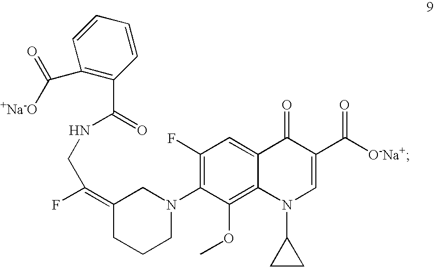

(k) converting

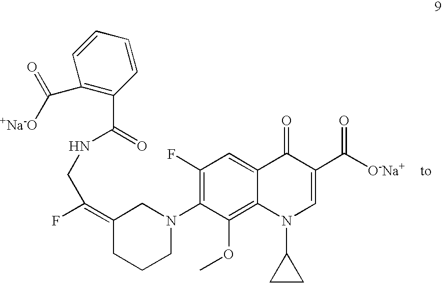

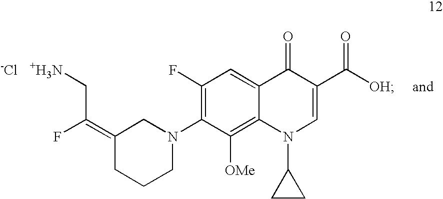

(l) converting

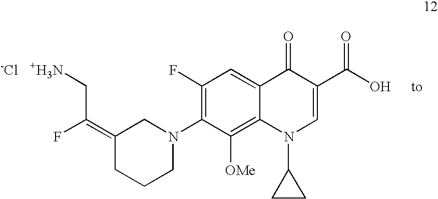

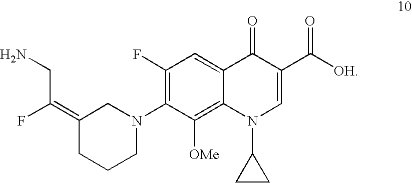

In one such example of this method of the invention, one or more extractions using one or more solvents selected from alcohol and non-polar aprotic can be performed in step (d). Particularly, the solvent is selected from 2-propanol, 2-MeTHF, toluene, diethyl ether, ethyl acetate, MTBE, and n-butanol. More particularly, the solvents are 2-MeTHF and toluene. More particularly, the solvent is n-butanol. More particularly, one extraction with 2-MeTHF and toluene is performed followed by another extraction with n-butanol.

### Chemical Definitions

As used herein, the following terms have the following meanings.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

The term “substituted” means one or more hydrogen atoms on a core molecule have been replaced with one or more radicals or linking groups, wherein the linking group, by definition is also further substituted. The substituent nomenclature used in the disclosure of the present invention was derived using nomenclature rules well known to those skilled in the art (e.g., IUPAC).

With reference to substituents, the term “independently” means that when more than one of such substituent is possible, such substituents may be the same or different from each other.

The term “dependently selected” means one or more substituent variables are present in a specified combination (e.g. groups of substituents commonly appearing in a tabular list).

The term “alkyl” means a saturated aliphatic straight, branched or cyclic-chain monovalent hydrocarbon radical or linking group substituent having from 1-10 carbon atoms, wherein the radical is derived by the removal of one hydrogen atom from a carbon atom and the linking group is derived by the removal of one hydrogen atom from each of two carbon atoms in the chain. The term includes, without limitation, methyl, methylene, ethyl, ethylene, propyl, propylene, isopropyl, isopropylene, n-butyl, n-butylene, t-butyl, t-butylene, pentyl, pentylene, hexyl, hexylene, cyclopentyl, cyclohexyl, and the like. An alkyl substituent may be attached to a core molecule via a terminal carbon atom or via a carbon atom within the chain. Similarly, any number of substituent variables may be attached to an alkyl substituent when allowed by available valences. The term “lower alkyl” means an alkyl substituent having from 1-4 carbon atoms.

The term “alkenyl” means an unsaturated or partially unsaturated hydrocarbon radical or linking group substituent having at least two carbon atoms and one double bond derived by the removal of one hydrogen atom from each of two adjacent carbon atoms in the chain. Atoms may be oriented about the double bond in either the E or Z configuration. The term includes, without limitation, methylidene, vinyl, vinylidene, allyl, propylidene, isopropenyl, iso-propylidene, prenyl, prenylene (3-methyl-2-butenylene), methallyl, methallylene, allylidene (2-propenylidene), crotylene (2-butenylene), and the like. An alkenyl substituent may be attached to a core molecule via a terminal carbon atom or via a carbon atom within the chain. Similarly, any number of substituent variables may be attached to an alkenyl substituent when allowed by available valences. The term “lower alkenyl” means an alkenyl substituent having from 2-4 carbon atoms.

The term “alkynyl” means an unsaturated or partially unsaturated hydrocarbon radical or linking group substituent having at least two carbon atoms and one triple bond derived by the removal of two hydrogen atoms from each of two adjacent carbon atoms in the chain. The term includes, without limitation, ethynyl, ethynylidene, propargyl, propargylidene and the like. An alkynyl substituent may be attached to a core molecule via a terminal carbon atom or via a carbon atom within the chain. Similarly, any number of substituent variables may be attached to an alkynyl substituent when allowed by available valences. The term “lower alkynyl” means an alkynyl substituent having from 2-4 carbon atoms.

The term “alkoxy” means an alkyl, alkenyl, or alkynyl radical or linking group substituent attached through an oxygen-linking atom, wherein a radical is of the formula —O-alkyl, —O-alkenyl, or —O-alkynyl, and a linking group is of the formula —O-alkyl-, —O-alkenyl-, or —O-alkynyl-. The term includes, without limitation, methoxy, ethoxy, propoxy, butoxy and the like. An alkoxy substituent may be attached to a core molecule and further substituted where allowed.

The term “cycloalkyl” means a saturated or partially unsaturated monocyclic, polycyclic or bridged hydrocarbon ring system radical or linking group. A ring of 3 to 10 carbon atoms may be designated by C₃₋₂₀ cycloalkyl; a ring of 3 to 12 carbon atoms may be designated by C₃₋₁₂ cycloalkyl, a ring of 3 to 8 carbon atoms may be designated by C₃₋₈ cycloalkyl and the like. The term “cycloalkyl” includes, without limitation, cyclopropyl, cyclobutyl, cyclopentyl, cyclohexyl, cyclohexenyl, cycloheptyl, cyclooctyl, indanyl, indenyl, 1,2,3,4-tetrahydro-naphthalen-2-yl, 5,6,7,8-tetrahydro-naphthalen-6-yl, 6,7,8,9-tetrahydro-5H-benzocyclohepten-6-yl, 5,6,7,8,9,10-hexahydro-benzocycloocten-6-yl, fluorenyl, bicyclo[2.2.1]heptyl, bicyclo[2.2.1]heptenyl, bicyclo[2.2.2]octyl, bicyclo[3.1.1]heptyl, bicyclo[3.2.1]octyl, bicyclo[2.2.2]octenyl, bicyclo[3.2.1]octenyl, adamantanyl, octahydro-4,7-methano-1H-indenyl, octahydro-2,5-methano-pentalenyl (also referred to as hexahydro-2,5-methano-pentalenyl) and the like. A cycloalkyl substituent may be attached to a core molecule and further substituted where allowed.

The term “aryl” means an unsaturated, conjugated 7c electron monocyclic or polycyclic hydrocarbon ring system radical or linking group substituent of 6, 9, 10 or 14 carbon atoms. The term includes, without limitation, phenyl, naphthalenyl, azulenyl, anthracenyl and the like. An aryl substituent may be attached to a core molecule and further substituted where allowed. In addition, the term “Ph” or “PH” refers to phenyl.

The term “heterocyclyl” means a saturated or partially unsaturated (such as those named with the prefix dihydro, tetrahydro, hexahydro and the like) monocyclic, polycyclic or bridged hydrocarbon ring system radical or linking group substituent, wherein at least one ring carbon atom has been replaced with one or more heteroatoms independently selected from N, O and S. A heterocyclyl substituent further includes a ring system having up to 4 nitrogen atom ring members or a ring system having from 0 to 3 nitrogen atom ring members and 1 oxygen or sulfur atom ring member. Alternatively, up to two adjacent ring members may be a heteroatom, wherein one heteroatom is nitrogen and the other is selected from N, O and S. A heterocyclyl radical is derived by the removal of one hydrogen atom from a single carbon or nitrogen ring atom. A heterocyclyl linking group is derived by the removal of one hydrogen atom from two of either a carbon or nitrogen ring atom. A heterocyclyl substituent may be attached to a core molecule by either a carbon atom ring member or by a nitrogen atom ring member and further substituted where allowed.

The term “heterocyclyl” includes, without limitation, furanyl, thienyl, 2-pyrrolinyl, 3-pyrrolinyl, pyrrolidinyl, pyrrolyl, 1,3-dioxolanyl, oxazolyl, thiazolyl, imidazolyl, 2-imidazolinyl (also referred to as 4,5-dihydro-1H-imidazolyl), imidazolidinyl, 2-pyrazolinyl, pyrazolidinyl, pyrazolyl, triazolyl, tetrazolyl, tetrazolinyl, tetrazolidinyl, 2H-pyranyl, 4H-pyranyl, thiopyranyl, pyridinyl, piperidinyl, 1,4-dioxanyl, morpholinyl, 1,4-dithianyl, thiomorpholinyl, pyridazinyl, pyrimidinyl, pyrazinyl, piperazinyl, azetidinyl, azepanyl, indolizinyl, indolyl, 4-aza-indolyl (also known as 1H-pyrrolo[3,2-b]pyridinyl, 6-aza-indolyl (also referred to as 1H-pyrrolo[2,3-c]pyridinyl), 7-aza-indolyl (also known as 1H-pyrrolo[2,3-b]pyridinyl, isoindolyl, indolinyl, benzo[b]furanyl, furo[2,3-b]pyridin-3-yl, benzo[b]thienyl, indazolyl (also referred to as 1H-indazolyl), benzoimidazolyl, benzothiazolyl, purinyl, 4H-quinolizinyl, quinolinyl, isoquinolinyl, cinnolinyl, quinazolinyl, quinoxalinyl, 1,8-naphthyridinyl, pteridinyl, quinuclidinyl, 2H-chromenyl, 3H-benzo[f]chromenyl, tetrahydro-furanyl, tetrahydro-thienyl, tetrahydro-pyranyl, tetrahydro-thiopyranyl, tetrahydro-pyridazinyl, hexahydro-1,4-diazepinyl, hexahydro-1,4-oxazepanyl, 2,3-dihydro-benzo[b]oxepinyl, 1,3-benzodioxolyl (also known as benzo[1,3]dioxolyl), 2,3-dihydro-1,4-benzodioxinyl (also known as benzo[1,4]dioxinyl), benzo-dihydro-furanyl (also known as 2,3-dihydro-benzofuranyl), benzo-tetrahydro-pyranyl, benzo-dihydro-thienyl, 2-aza-bicyclo[2.2.1]heptyl, 1-aza-bicyclo[2.2.2]octyl, 8-aza-bicyclo[3.2.1]octyl, 7-oxa-bicyclo[2.2.1]heptyl, pyrrolidinium, piperidinium, piperazinium, morpholinium and the like. Preferably, “heterocyclyl” as used herein includes pyridyl, thiophene, oxazole, isoxazole, and thiazole. More preferably, a “heterocyclyl” is pyridyl.

The term “acyl” means a radical of the formula —C(O)-alkyl, —C(O)-alkenyl, —C(O)-alkynyl, or a linking group of the formula —C(O)-alkyl-, —C(O)-alkenyl-, or —C(O)-alkynyl-.

The term “halo” or “halogen” means fluoro (F), chloro (Cl), bromo (Br), or iodo (I).

The term “base” means a chemical species or molecular entity having an available pair of electrons capable of forming a covalent bond with a hydron (proton) or with the vacant orbital of some other species.

The present invention also contemplates preparing compounds of Formula (1) in various stereoisomeric or tautomeric forms, including those in the form of essentially pure enantiomers, racemic mixtures or tautomers.

The term “isomer” means compounds that have the same composition and molecular weight but differ in physical and/or chemical properties. Such substances have the same number and kind of atoms but differ in structure. The structural difference may be in constitution (geometric isomers) or may result in an ability to rotate the plane of polarized light (stereoisomers).

The term “stereoisomer” means isomers of identical constitution that differ in the arrangement of their atoms in space. Enantiomers and diastereomers are stereoisomers wherein an asymmetrically substituted carbon atom acts as a chiral center. The term “chiral” refers to a molecule that is not superposable on its mirror image, implying the absence of an axis and a plane or center of symmetry. The term “enantiomer” refers to one of a pair of molecular species that are mirror images of each other and are not superposable. The term “diastereomer” refers to stereoisomers that are not related as mirror images. The symbols “R” and “S” represent the configuration of substituents around a chiral carbon atom(s). The symbols “R*” and “S*” denote the relative configurations of substituents around a chiral carbon atom(s).

The term “racemate” or “racemic mixture” means a compound of equimolar quantities of two enantiomeric species, wherein the compound is devoid of optical activity. The term “optical activity” refers to the degree to which a chiral molecule or nonracemic mixture of chiral molecules rotates the plane of polarized light.

The term “geometric isomer” as used herein means isomers that differ in the orientation of substituent atoms in relationship to a carbon-carbon double bond, to a cycloalkyl ring, or to a bridged bicyclic system. Substituent atoms (other than H) on each side of a carbon-carbon double bond may be in an E or Z configuration.

An isomer is designated as being in the “Z” (zusammen=“together”) configuration if the groups of highest priority lie on the same side of a reference plane passing through the double bond and perpendicular to the plane containing the bonds linking the groups to the double-bonded atoms; the other isomer is designated as “_” (entgegen=“opposite”). The term “priority” used to determine E and Z isomers herein refers to the rules established for the purpose of unambiguous designation of isomers described in R. S. Cahn, C. K. Ingold and V. Prelog, Angew. Chem. 78, 413-447 (1966); Angew. Chem. Internat. Ed. Eng. 5, 385-415, 511 (1966); and V. Prelog and G. Helmchen, Angew. Chem. 94, 614-631 (1982), Angew. Chem. Internat. Ed. Eng. 21, 567-583 (1982). Certain products of the synthetic methods of the present invention are isomeric alcohols in such E or Z configuration.

Substituent atoms (other than H) attached to a hydrocarbon ring may, in some cases, also be referred to be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”. Substituent atoms (other than H) attached to a bridged bicyclic system may be in an “endo” or “exo” configuration. In the “endo” configuration, the substituents attached to a bridge (not a bridgehead) point toward the larger of the two remaining bridges; in the “exo” configuration, the substituents attached to a bridge point toward the smaller of the two remaining bridges.

In particular, the term “isomeric alcohols of Formula (1)” refers to a mixture of E and Z-isomers of compounds of Formula (1)

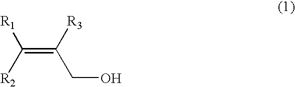

wherein
R¹ and R² are different groups selected from H, C₁₋₁₀alkyl, C₂₋₁₀alkenyl, and C₂₋₁₀alkynyl, or R¹, R² and the C atom they attach to may together form an asymmetric group selected from substituted C₃₋₁₀cycloalkyl and optionally substituted heterocyclyl; and
R³ is H, unsubstituted C₁₋₁₀alkyl, halogen, aryl, or heterocyclyl.

It is to be understood that the various substituent stereoisomers, geometric isomers and mixtures thereof used to perform the methods of the present invention are either commercially available, can be prepared synthetically from commercially available starting materials, or can be prepared as isomeric mixtures and then obtained as resolved isomers using techniques well-known to those of ordinary skill in the art. Conventional resolution techniques include forming the free base of each isomer of an isomeric pair using an optically active salt (followed by fractional crystallization and regeneration of the free base), forming an ester or amide of each of the isomers of an isomeric pair (followed by chromatographic separation and removal of the chiral auxiliary) or resolving an isomeric mixture of either a starting material or a final product using various well known chromatographic methods.

The isomeric descriptors “R,” “S,” “S*,” “R*,” “E,” “Z,” “cis,” “trans,” “exo”, and “endo”, where used herein, indicate atom configurations relative to a core molecule and are intended to be used as defined in the literature.

During any of the processes according to the present invention for preparation of compounds of Formula (1), it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

### Synthetic Schemes

Representative methods of the present invention are shown in the general synthetic scheme(s) described below and are illustrated more particularly in the specific examples that follow. The general schemes and specific examples are offered by way of illustration; the invention should not be construed as being limited by the chemical reactions and conditions expressed herein. The methods for preparing the various starting materials used in the schemes and examples are well within the skill of persons versed in the art.

The following abbreviations and formulas have the indicated meanings:
Ac CH₃(CO)—
Ac₂O acetic anhydride
Boc tert-butoxy carbonyl or t-butoxy carbonyl
CH₂Cl₂ or DCM methylene chloride or dichloromethane
CHCl₃ chloroform
CH₃CN or acetonitrile
MeCN
Cpd or cmpd compound
DABCO 1,4-diazabicyclo[2.2.2]octane
DBU 1,8-diazabicyclo[5.4.0]undec-7-ene
DIAD diisopropyl azodicarboxylate
DIBAL diisobutyl aluminum hydride
DIPEA diisopropylethylamine
DMAP 4-dimethylaminopyridine
DME dimethoxyethane
DMF N,N-dimethyl formamide
EDCI 1-(3-dimethylaminopropyl)-3-ethylcarbodiimide hydrochloride
Et ethyl
Et₂O diethyl ether
EtOAc or ethyl acetate
CH₃CO₂Et
HPLC High Performance Liquid Chromatography
IPA 2-propanol
LAH or LiAlH₄ lithium aluminum hydride
LC-MS analysis method combining HPLC and mass spectrometry
LHMDS lithium bis(trimethylsilyl)amide
LiOH lithium hydroxide
Me methyl
MeOH/CH₃OH methanol
MsCl methanesulfonyl chloride
MTBE methyl tert-butyl ether
min(s)/h(s), minute(s)/hour(s)/day(s)
hr(s)/d(s)
MS mass spectrum, refers to data shown as m/z (M+H)+
NH₄Cl ammonium chloride
N(i-Pr)₂Et diisopropylethylamine
NaH sodium hydride
NaHCO₃ sodium bicarbonate
NaN₃ sodium azide
NaOH sodium hydroxide
Na₂SO₄ sodium sulfate
NMR nuclear magnetic resonance spectroscopy
psi pounds per square inch
PTLC preparative thin layer chromatography
RT/rt/r.t. room temperature
SOCl₂ thionyl chloride
TEA or Et₃N triethylamine
TFA trifluoroacetic acid
THF tetrahydrofuran
TMSCl chlorotrimethylsilane or trimethylsilyl chloride

According to Scheme 3 below, an example of the present invention is to combine the phosphonate, solvent, and ketone or aldehyde in one reaction vessel followed by addition of a reducing agent, as depicted in the following reactions:

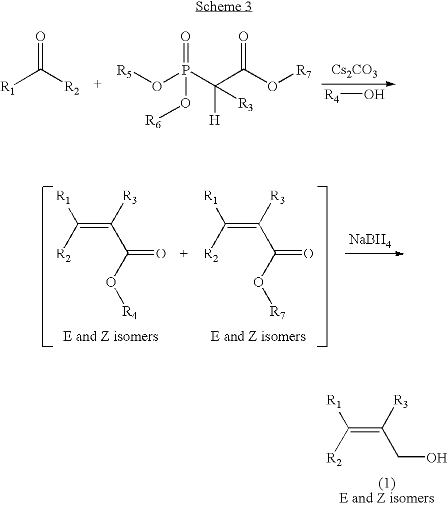

As shown in Scheme 3, wherein R₄ represents C₁₋₁₀alkyl or aryl, and R₁, R₂, R₃, R₅, R₆, and R₇ are as described above, the solvent is preferably, but not limited to, one or more alcohols having 1-6 carbon atoms such as 2-methoxyethanol and ethanol. A base, preferably Cs₂CO₃, is added as a solid or in solution to the reaction mixture. After the formation of the ester is complete, a reducing agent, preferably NaBH₄, is added to the reaction mixture without any isolation. After the reduction step is complete (often in 1-30 hours), the reaction mixture is diluted with water. The aqueous mixture is next extracted with an organic solvent to provide the desired product.

In the case where this method is applied to the preparation of alcohol 2′,

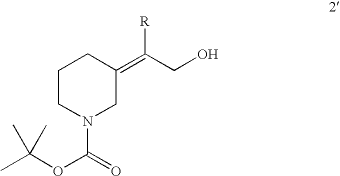

wherein R is H, alkyl, halogen, aryl, or heterocyclyl, alcohol 2′ has usually been separated via column chromatography into the individual isomers 2′a and 2′b,

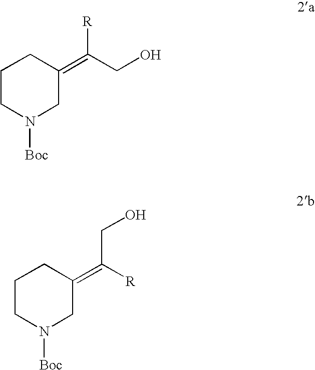

as noted hereinabove. According to this invention, however, the reaction steps are conducted in one reaction vessel and the separation step obviates the need for column chromatography.

Alternatively, as shown in Scheme 4, wherein

R¹, R² and the C atom they attach to together form

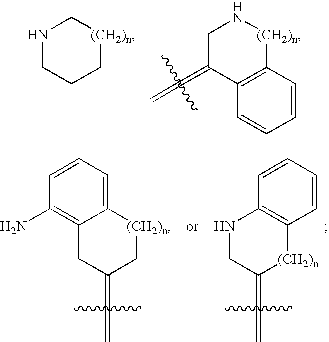

R³ is H, unsubstituted C₁₋₁₀alkyl, halogen, aryl, or heterocyclyl;

n is 0-4; and

R₄, R₅, R₆, and R₇ are as described above, isomeric alcohols of Formula (1) can be further converted into isomers of Formula (2), which can then be separated via selective crystallization utilizing, for instance, 5-6N HCl in 2-propanol, in the form of their respective salts.

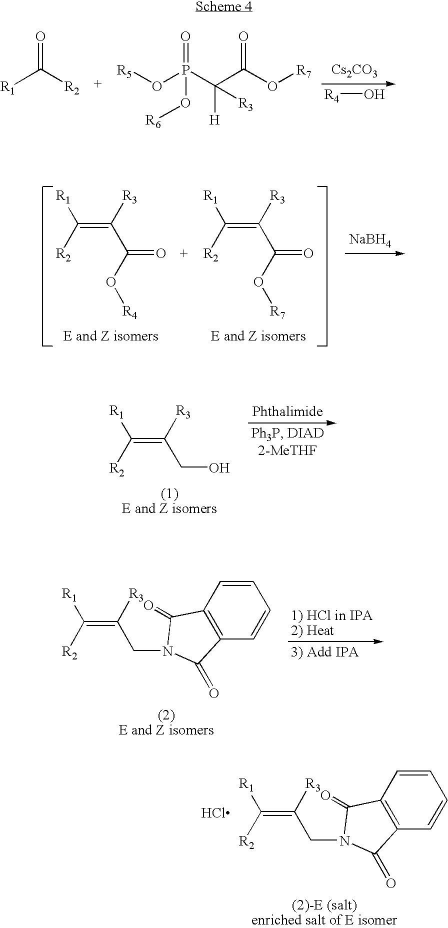

The invention is further defined by reference to the following examples, which are merely intended to be illustrative and not limiting.

### EXAMPLE 1

### Preparation of 7-[3-(2-Amino-1-fluoroethylidene)piperidin-1-yl]-1-cyclopropyl-6-fluoro-8-methoxy-4-oxo-1,4-dihydroquinoline-3-carboxylic acid (10) and its HCl salt (12)

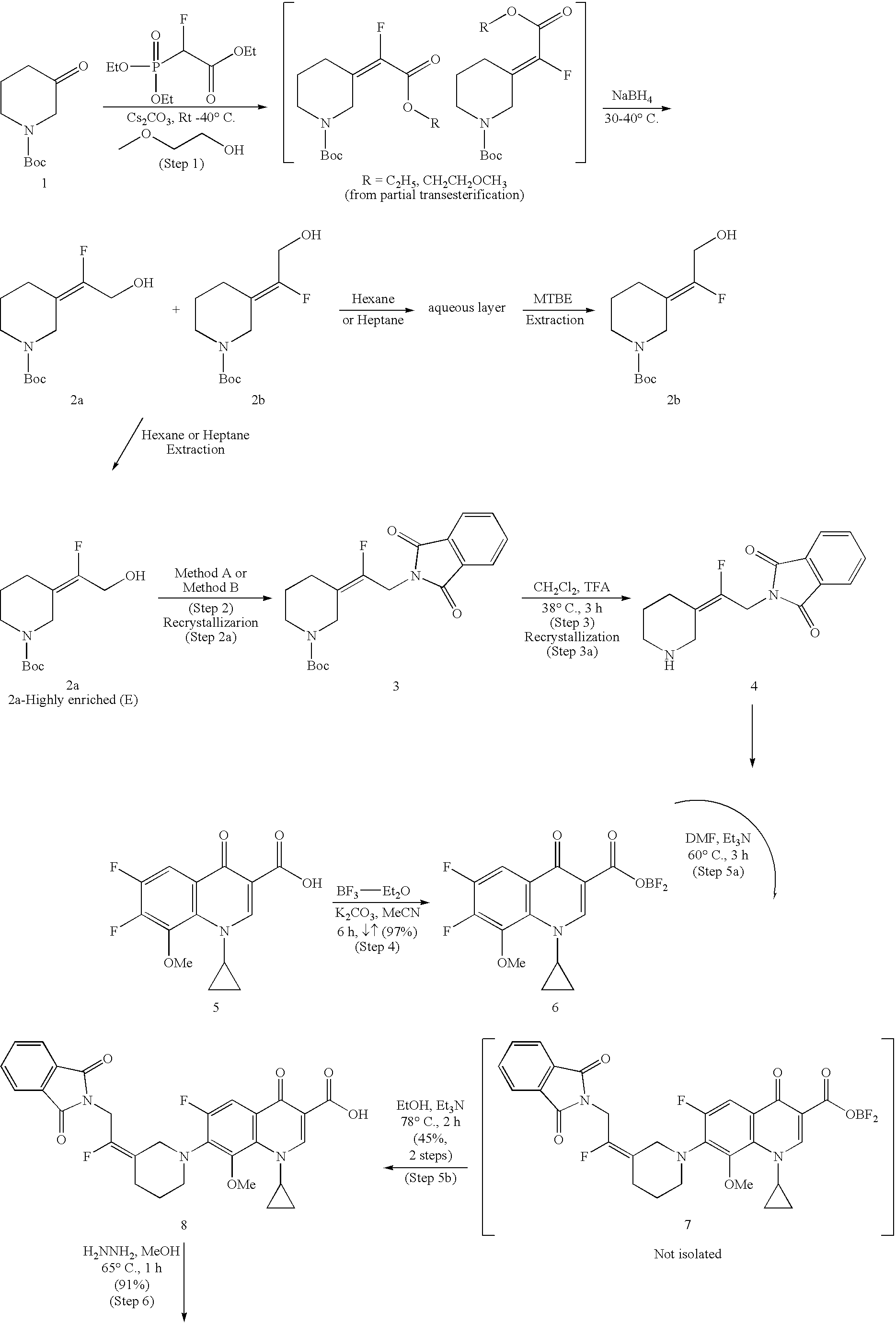

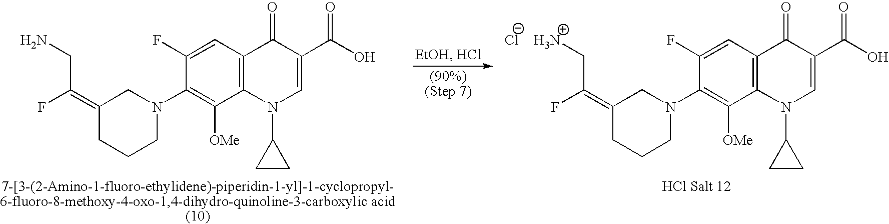

### Step 1: Preparation of 3-(1-fluoro-2-hydroxyethylidene)piperidine-1-carboxylic acid tert-butyl ester (2a)

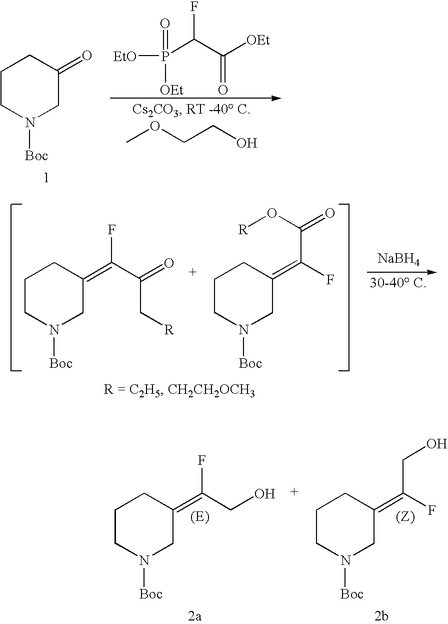

A 22-L 4-neck round bottom flask, equipped with a thermocouple controller, overhead mechanical stirrer, condenser, nitrogen inlet adapter, and stopper, was charged with N-Boc-3-piperidone (663.36 g, 3.34 mol), 2-methoxyethanol (6.0 L) and 2-fluorotriethylphosphonoacetate (843.54 g, 3.49 mol). The mixture was stirred to obtain a homogeneous solution and then Cs₂CO₃ was added in portions over 1.5 h. After the Cs₂CO₃ addition was complete, NaBH₄ was added in portions over 6 h; during most of this addition the reaction temperature was maintained between 35° C. to 40° C. After the addition was complete, the reaction was allowed to stir overnight after which time HPLC analysis indicated that the reaction was complete. This run was combined with two additional runs of equal size and transferred to a stirred 100-L Hastalloy® reactor containing water (90 L). The aqueous mixture was extracted with heptane (4×20 L) followed by extraction with MTBE (methyl tert-butyl ether) (20 L). The first three heptane extracts provided 842 g of the allylic alcohol as 71:29 (E:Z) mixture (HPLC and NMR). The product mixture from the first three heptane extractions was carried on to the next step without any additional purification. The fourth heptane extract gave 114 g of product that was a 67:33 mixture of E:Z alcohols (NMR). MTBE extraction and concentration gave 1.1 Kg of product as a 33:67 mixture of E:Z alcohols (HPLC). The total overall yield for both isomers was 2.06 Kg (83%). ¹H NMR of 2a (400 MHz, CDCl₃): 1.45 (s, 9H), 1.52 (m, 2H), 2.40 (m, 2H), 3.45 (m, 2H), 3.90 (s, 2H), 4.25 (d, 2H). ¹H NMR of 2b (400 MHz, CDCl₃): δ 1.46 (s, 9H), 1.65 (m, 2H), 2.27 (m, 2H), 3.45 (m, 2H), 4.1 (s, 2H), 4.25 (d, 2H).

### Step 2, Method A: Preparation of 3-E-[2-(1,3-dioxo-1,3-dihydroisoindol-2-yl)-1-fluoroethylidene]-piperidine-1-carboxylic acid tert-butyl ester (3-E)

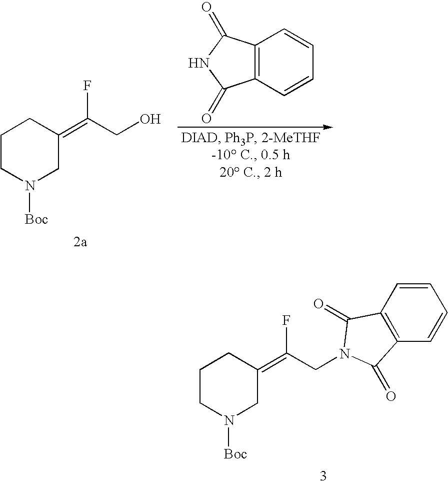

A 22-L 4-neck round bottom flask, equipped with a thermocouple controller, overhead mechanical stirrer, condenser, pressure-equalizing addition funnel, nitrogen inlet adapter, and stopper, was charged with E:Z alcohol mixture 2a and 2b (377.5 g, 1.296 mol corrected), 2-MeTHF (3.31 L), phthalimide (232.8 g, 1.581 mol), and Ph₃P (411.3 g, 1.568 mol). The white suspension was stirred under N₂ and cooled to −12° C. in an acetone/Dry-Ice bath, DIAD (309 mL, 1.49 mol) was added via the addition funnel over a 36-min period, while the reaction temperature was maintained at −15° C. to −10° C. After the addition, the reaction was warmed to 20° C. in a water bath and stirred for 2 h. The reaction was cooled to 0° C. in an ice/water bath and quenched with cold 1.0 M HCl (950 mL). The aqueous phase was separated and EtOAc (1.70 L) was added to the organic phase. This phase was washed with cold 1.0 M HCl (0.95 L) (the aqueous phase was pH≦2) and then separated. The organic phase was next washed with cold 4 NNaOH (1.70 L), the alkaline aqueous phase (pH≧13) was separated and the EtOAc layer washed with brine (1.70 L). Concentration of the organic phase at 60° C. under house vacuum (˜120 mm Hg) afforded 1,442.0 g of crude 3. This run was repeated on the same scale to provide an additional 1,431.0 g of crude material for a combined yield of 2,873 g (159%). HPLC analysis (area %) indicated crude 3 was a mixture of 3-E (29.4%), 3-Z (10.4%), Ph₃PO (51.0%), and phthalimide (1.1%). This was purified by recrystallization as described in step 2a.

Step 2a, Method A: Purification of 3-E-[2-(1,3-dioxo-1,3-dihydroisoindol-2-yl)-1-fluoroethylidene]-piperidine-1-carboxylic acid tert-butyl ester

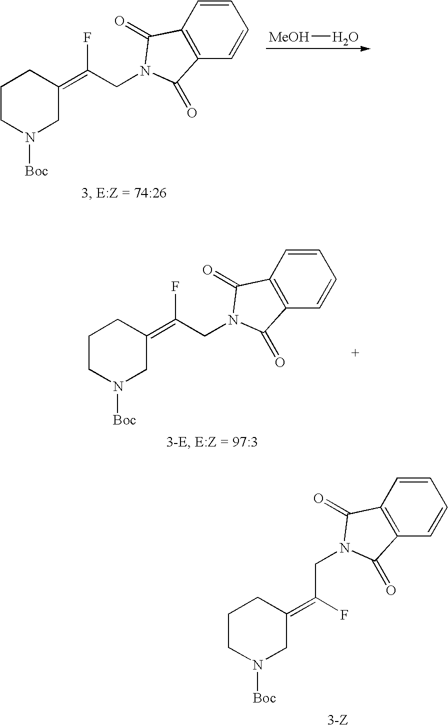

A 22-L 4-neck round bottom flask equipped with a thermocouple controller, overhead mechanical stirrer, condenser, pressure-equalizing addition funnel, nitrogen inlet adapter and stopper was charged with the combined crude 3 (2,873 g) and MeOH (9.0 L). The solution was stirred under nitrogen and heated to 65° C., while hot (60° C.)

D.I. water (7.8 L) was added over a 15-min period. The solution was stirred at 65° C. for 5 min, and then the heating mantle was replaced with a water bath, and the mixture was gradually cooled to 0° C. over a 4-h period, and continued stirring for 1 h at 0° C. The off-white solid was collected by filtration, and dried by air-suction at 60° C. for 20 h, this provided 1,172.6 g of a mixture of 3-E and 3-Z.

The partially purified product above was recrystallized a second time in the same manner using hot MeOH (7.2 L) and hot water (5.0 L) except that the water was added over a 10-min period to afford 515.6 g (53.4%) of 3-E as a 97:3 mixture of E:Z geometric isomers. This material was used in the next step without additional purification. ¹H NMR of 3-E (400 MHz, CDCl₃): δ 1.48 (s, 9H), 1.52-1.66 (m, 2H), 2.28-2.38 (m, 2H), 3.40-3.51 (m, 2H), 4.18 (s, 2H), 4.55 (d, J=21.0 Hz, 2H), 7.68-7.77 (m, 2H), 7.80-7.89 (m, 2H). MS: 397 (M+Na)⁺, 771 (2M+Na)⁺.

3-E-[2-(1,3-dioxo-1,3-dihydroisoindol-2-yl)-1-fluoroethylidene]-piperidine-1-carboxylic acid tert-butyl ester was also prepared with Method B below:

Step 2, Method B: Preparation of 3-E-[2-(1,3-dioxo-1,3-dihydroisoindol-2-yl)-1-fluoroethylidene]-piperidine-1-carboxylic acid tert-butyl ester (3-E)

### Preparation of the Methanesulfonate and Chloride Derivatives

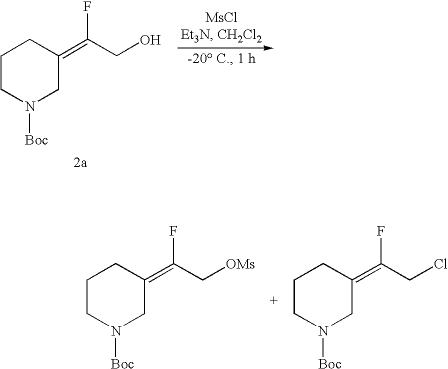

A 12-L 4-neck round bottom flask equipped with an overhead stirrer, thermocouple, pressure-equalizing addition funnel, and a nitrogen inlet adapter was charged with 2a (297.0 g, 1.21 mol) and CH₂Cl₂ (3.9 L). The solution was cooled to 0° C. under N₂ and Et₃N (320 mL, 2.30 mol) was added via the addition funnel over a 10-min period. This was followed by methanesulfonyl chloride (115 mL, 1.49 mol) added over a 60-min period then the reaction was stirred for an additional 60-min at 0° C. The mixture was poured into a mixture of deionized water (4.4 L) and saturated NaHCO₃ (0.78 L), the layers were separated, the aqueous layer was extracted with CH₂Cl₂ (2×2 L). All the CH₂Cl₂ layers were combined and washed with saturated NaHCO₃ (2 L). The CH₂Cl₂ was removed under vacuum at 40° C. to afford a mixture of the mesylate and chloride (342.3 g). This mixture was taken on to the next step without any purification.

Conversion of the methanesulfonate/chloride to phthalimide 3

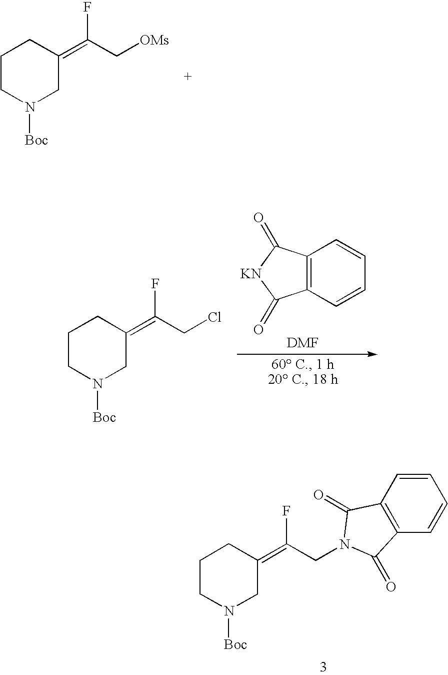

A 5-L 4-neck round bottom flask equipped with an overhead stirrer, thermocouple, pressure-equalizing addition funnel, and a nitrogen inlet adapter was charged with the mixture of the mesylate and chloride from above (342.2 g, 1.21 mol) and DMF (2.0 L) followed by potassium phthalimide (224.9 g, 1.21 mol). The mixture was stirred at 60° C. for 1-h then at 20° C. for 18 h. The mixture was poured into ice-water, allowed to stand for 30-min and filtered. The liquors from the filtration were allowed to stand at 0° C. over the weekend and filtered again. The combined solids were dissolved in acetone (4 L) and concentrated on the rotary evaporator, this process was repeated a second time to give the phthalimide derivative 3 as a mixture of E/Z (79/31) isomers (263.2 g, 58.1%).

Step 2a, Method B: Purification of 3-E-[2-(1,3-dioxo-1,3-dihydroisoindol-2-yl)-1-fluoroethylidene]-piperidine-1-carboxylic acid tert-butyl ester

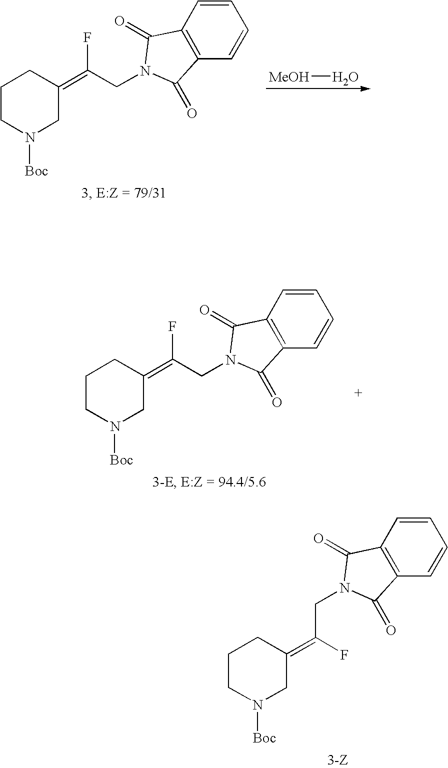

A 12-L 4-neck round bottom flask equipped with an overhead stirrer, thermocouple, pressure-equalizing addition funnel, and a nitrogen inlet adapter was charged with the crude phthalimide derivative 3 (263.1 g) and MeOH (2.74 L). The mixture was heated to 66-68° C. while water (2.1 L) was added over 20-min, the mixture was stirred at 68° C. for 5-min, then gradually cooled to 20° C. for 18-h. While the crystallization mixture was cooling it was seeded at 60° C., 56° C. and 53° C. This crystallization gave a white solid that was filtered and dried under vacuum at 50° C. to afford 3-E (118.8 g, 45.2%) as a mixture containing 94.4% E and 5.6% Z isomers (NMR analysis).

### Step 3: Preparation of 2-[2-fluoro-2-(3-piperidinylidene)ethyl]-1H-isoindole-1,3)-dione (4)

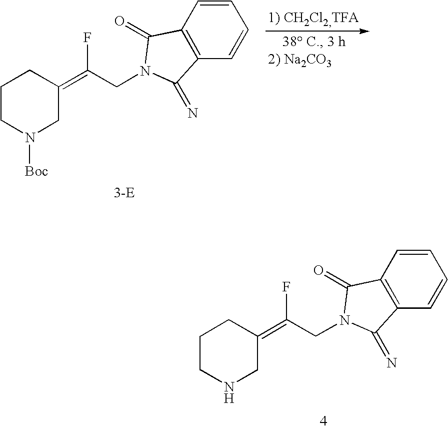

A 12-L 4-neck round bottom flask equipped with an overhead stirrer, thermocouple, pressure-equalizing addition funnel, and a nitrogen inlet adapter was charged with 3-E (578.0 g, 1.544 mol) and CH₂Cl₂ (4.5 L). The solution was stirred at 20° C. under N₂ and TFA (476 mL, 6.18 mol) was added via the addition funnel over a 10-min period. The mixture was gently heated to 38° C. and stirred for 3 h. The solvent was removed under vacuum to give the TFA salt of 4 (962.6 g). This material was dissolved in CH₂Cl₂ (4.0 L) and washed with 2.5 NNa₂CO₃ (4.6 L)-followed by saturated NaHCO₃ (4.6 L). The organic phase was dried (MgSO₄), filtered, and condensed in vacuo. The off-white solid was dried at 40° C. under vacuum (20 mm Hg) for 20 h to afford 464.3 g of the free base of 4 as slightly yellowish foamy substance.

¹H NMR of 4 TFA salt (400 MHz, CDCl₃): δ 1.87-1.98 (m, 2H), 2.42-2.55 (m, 2H), 3.38-3.50 (m, 2H), 4.08-4.18 (br s, 2H), 4.50 (d, J=21.0 Hz, 2H), 7.69-7.78 (m, 2H), 7.79-7.87 (m, 2H), 7.98-8.23 (br s, 1H), 12.48 (s, 1H). MS: 275 (MH)⁺, 549 (2M+H)⁺.

### Step 4: Preparation of 1-Cyclopropyl-6,7-difluoro-8-methoxy-4-oxo-1,4-dihydroquinoline-3-carboxylic acid difluoroborate ester (6)

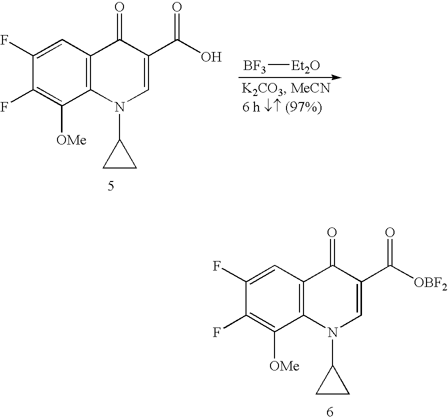

A 22-L 4-neck round bottom flask equipped with an overhead stirrer, thermocouple, condenser, pressure equalizing addition funnel, and a nitrogen inlet adapter was charged with quinoline-3-carboxylic acid 5 (450.0 g, 1.524 mol), THF (5.40 L) and K₂CO₃ (247.2 g, 1.753 mol). This suspension was first stirred at 20° C. under N₂ for 5 min, and BF₃•Et₂O (259 mL, 2.04 mol) was added dropwise via the addition funnel to the stirred mixture over a 5-min period. After the addition, the mixture was heated to reflux (66° C.) for 6 h. The reaction was cooled to 10° C., diluted with Et₂O (9.0 L) and stirred for 10 min. The solid was filtered and washed with Et₂O (200 mL×2) and then dried at 50° C. under house vacuum (˜160 mm Hg) for 20 h to afford 771.0 g of crude difluoroborate ester 6. After this, the crude material was suspended in MeCN (8.0 L) and stirred at 20° C. for 20 min; the solid was collected by filtration. The filter cake was re-suspended and stirred in MeCN four more times (2.0 L×4), and all filtrates were combined and concentrated at 60° C. under hi-vac (˜10 mmHg). The resulting off-white solid was dried at 50° C. under house vacuum (˜160 mmHg) for 20 h to afford 508.66 g (97.2% isolated yield, HPLC=99.2% by area) of pure difluoroborate ester 6. ¹H NMR of 6 (400 MHz, CD₃CN): δ1.17-1.28 (m, 2H), 1.29-1.40 (m, 2H), 4.19 (s, 3H), 4.40-4.52 (m, 1H), 8.16 (dd, J=6.9, 7.0 Hz, 1H), 9.17 (s, 1H). MS: 344 (MH)⁺, 667 (2M−F)⁺.

### Step 5: Preparation of intermediate 8

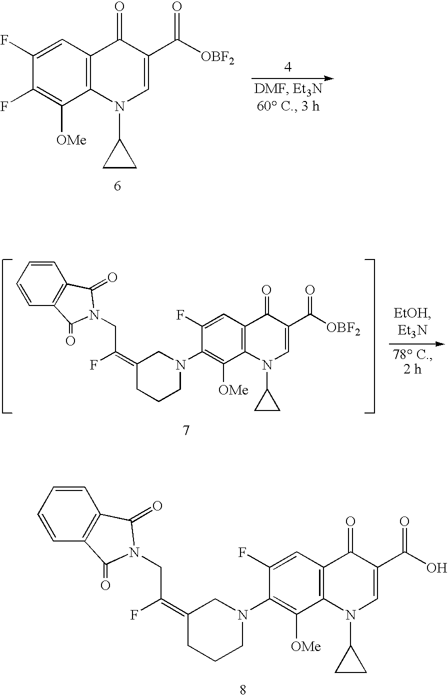

A 5-L 4-neck round bottom flask equipped with an overhead stirrer, thermocouple, condenser, pressure-equalizing addition funnel and a nitrogen inlet adapter was charged with difluoroborate ester 6 (320.0 g, 0.933 mol), DMF (1.10 L) and piperidine 4 (289.0 g, 1.053 mole). This suspension was stirred at 20° C. under N₂ for 5 min, Et₃N (299 mL, 2.15 mol) was added to the stirred mixture via the addition funnel over an additional 5-min period. After this addition, the mixture was heated to 60° C. and stirred for 3 h, to give crude intermediate 7. HPLC analysis (area %) indicated crude 7 is a mixture of 7 (40.5%), 8 (1.7%), 6 (24.1%), and the rest of unknowns (33.7%). MS: 598 (MH)⁺. The coupled crude product 7 was carried on to the next step without isolation.

### Removal of the Fluoroborate Ester

The above stirred reaction mixture containing 7 was treated in the same flask with EtOH (6.80 L) and Et₃N (299 mL, 2.147 mol) under N₂ at 60° C. The amber solution was heated to reflux at 72° C. for 2 h and cooled to 20° C. The reaction mixture was poured into a rapidly stirred 22-L 4-neck round bottom flask containing a 1:1 (v/v) ice-water mixture (8.0 L) over a 10-min period; stirring was continued for ˜10 min. Cold 1 NHCl (4.0 L) was added to the solution over 20 min to adjust the pH from 9-10 to 3; stirring was continued for an additional 20 min at 0° C. The yellow solid was isolated by filtration and dried in a filter funnel by air-suction using house vacuum (˜160 mm Hg) at 20° C. for 20 h to afford 1,889.0 g of crude 8 as a damp solid (HPLC=33.6%, area %).

### Purification of Intermediate 8

To a 22-L 4-neck round bottom flask equipped with an overhead stirrer, thermocouple, pressure-equalizing addition funnel, and a nitrogen inlet adapter was charged with crude 8 (1889.0 g), MeCN (3.6 L) and EtOH (3.2 L). The suspension was heated to reflux (76° C.), while D.I. H₂O (500 mL) was added over 10 min. The solution was stirred at 76° C. for 5 min, and then gradually cooled to 10° C. over 1 h; stirred for an additional hour. The yellow solid was collected by filtration, dried in a vacuum oven under house vacuum (˜160 mm Hg) at 60° C. for 20 h to afford 229.1 g (45%) of 8, which was used in next step without further purification. ¹H NMR of 8 (400 MHz, DMSO-d₆): δ1.02-1.10 (m, 2H), 1.11-1.19 (m, 2H), 1.67-1.79 (m, 2H), 2.34-2.45 (m, 2H), 3.38-3.49 (m, 2H), 3.78 (s, 3H), 4.10 (s, 2H), 4.15-4.26 (m, 1H), 4.54 (d, J=21.0 Hz, 1H), 7.72 (d, J=9.1 Hz, 1H), 7.81 (s, 4H), 8.71 (s, 1H), 14.98 (s, 1H). MS: 550 (MH)⁺.

### Step 6: Preparation of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (10)

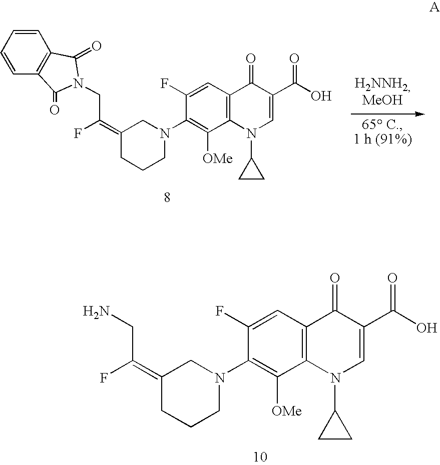

22-L 4-neck round bottom flask equipped with an overhead stirrer, thermocouple, condenser, pressure-equalizing addition funnel and a nitrogen inlet adapter was charged with 8 (253.6 g, 0.462 mol) and MeOH (5.10 L). This suspension was stirred at 20° C. under N₂ and H₂NNH₂ (86.9 mL, 2.796 mol) was added over a 5-min period. The yellow suspension was heated to 65° C. and refluxed for 1 h. The reaction was cooled to 60° C. and MeCN (3.84 L) was added. The mixture was heated to reflux for 5 min, and then cooled to 20° C. in a water bath. The light-yellow solid was collected by filtration and the filter cake was washed with MeCN (150 mL×2). The combined filtrate was concentrated at 60° C. affording 322.0 g of crude product 10. This product was recrystallized from a mixture of MeOH (1.0 L) and water (1.195 L) to give 176.6 g (91.2%) of pure product 10 as a light yellow solid. ¹H NMR of 10 (400 MHz, DMSO-d₆): δ1.0-1.09 (m, 2H), 1.10-1.19 (m, 2H), 1.66-1.78 (m, 2H), 2.30-2.41 (m, 2H), 3.17 (s, 2H), 3.35 (s, 1H), 3.36-3.47 (m, 2H), 3.74 (s, 3H), 3.89 (s, 2H), 4.13-4.22 (m, 1H), 5.35-6.18 (br, 2H), 7.74 (d, J=8.9 Hz, 1H), 8.69 (s, 1H). MS: 420 (MH)⁺.

### EXAMPLE 2

### Alternative Process to Prepare 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid (10)

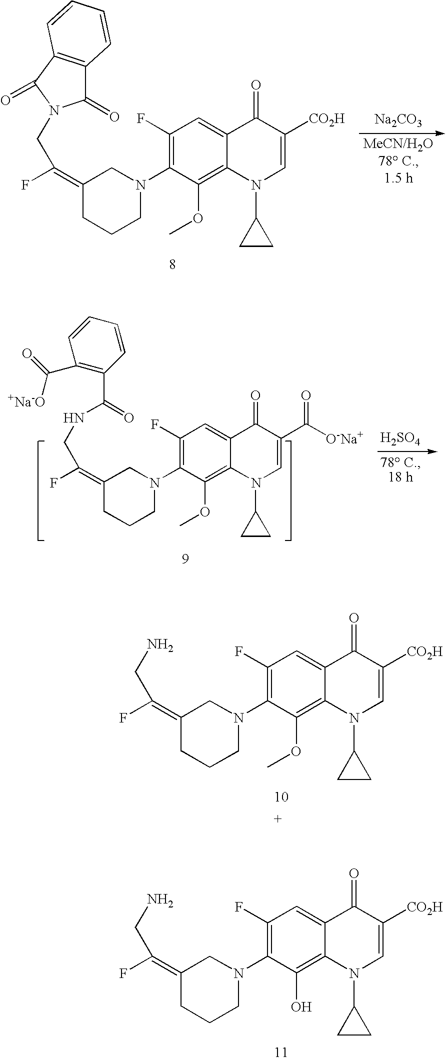

A 50-mL 3-neck round bottom flask equipped with a magnetic stirrer, a thermocouple, a condenser, a pressure-equalization dropping funnel, and a N₂ inlet adapter, was charged with phthalimide intermediate 8 (92.3%, 1.0 g, 1.82 mmol, 1.0 eq.), MeCN (1.5 mL), and H₂O (4.1 mL). This mixture was stirred at 20° C. under N₂, and a solution of 30% Na₂CO₃ (1.69 mL, 6.95 eq.) was added over a 2-min period, and then the mixture was heated to 78° C. and stirred for 3 h. The progress of the reaction was monitored by HPLC and LC-MS, both of which indicated that the compound 8 was completely converted to sodium dicarboxylate amide 9 (HPLC=91%, area %, solution yield, plus 0.4% of starting 8) after 90 min. No further changes were observed after the reaction was stirred for 3 h. MS of 9: MH⁺=590, M−Na⁺=566.

The reaction was cooled to 20° C. and MeCN (1.74 mL) was added, followed by the additions of a 50% solution of H₂SO₄ (1.4 mL, 3.92 eq.) and H₂O (0.67 mL). The mixture was again heated to 78° C. and stirred for 18 h. The progress of the reaction was monitored by HPLC and LC-MS, and both indicated that amide 9 was almost completely hydrolyzed to the product 10 (HPLC=89.5%, area %, solution yield, plus 0.2% of 9) after 2 h. The reaction was stirred at 78° C. for an additional 16 h, and then cooled to 20° C. Anhydrous EtOH (20 mL×2) was added to the mixture and concentrated twice at 60° C. under high vacuum (20 mmHg) to afford the crude product as a dark brown paste. HPLC analysis showed the mixture consisted of 26% (HPLC area %, solution yield) amine 10, 6.4% 8-demethylated 11 (MH⁺=406. HPLC retention time=3.03 min/11), 12.4% intermediate 9, 8.4% starting 8, and the rest of unknowns. The structures of compounds 9, 10 and 11 were all confirmed by comparing to the HPLC (retention time) and LC-MS of authentic samples.

### EXAMPLE 3

### Preparation of 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid hydrogen chloride salt (12)

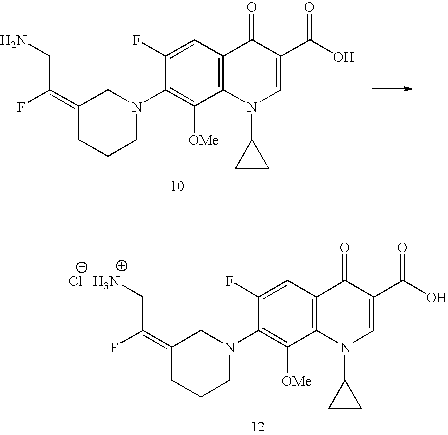

7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (10) was prepared as described in Step 6 of Example 1.

A 5-L 4-neck round bottom flask equipped with an overhead stirrer, thermocouple, condenser, pressure-equalizing addition funnel, and a nitrogen inlet adapter was charged with compound 10 (176.0 g, 0.4196 mol) and EtOH (2.40 L). The suspension was stirred under N₂ and cooled to 10° C. with an ice/water bath. A solution of HCl in EtOH (1.25 M, 350 mL) was added via the addition funnel over a 20-min period. After the addition, the reaction was stirred at 10° C. for 5 min. The water bath was replaced with a heating mantle and the solution was heated to 76° C. and stirred for 5 min. The heating mantle was replaced with the water bath, the solution was cooled to 0° C. over 1 h and stirred at this temperature for an additional 1 h. The solid was collected by filtration, washed with ice-cold EtOH (100 mL×2) and dried at 60° C. under vacuum (˜4 mmHg) for 60 h. There was obtained 88.9 g (82%) of HCl salt 12 as an off-white to very light-yellow solid. ¹H NMR of HCl salt 12 (400 MHz, CD₃CO₂D): δ1.10-1.19 (m, 2H), 1.29-1.38 (m, 2H), 1.81-1.93 (m, 2H), 2.51-2.60 (m, 2H), 3.48-3.60 (m, 2H), 3.86 (s, 3H), 4.08 (s, 2H), 4.18 (s, 1H), 4.19-4.30 (m, 2H), 7.92 (d, J=8.6 Hz, 1H), 8.98 (s, 1H) 11.65 (s, 1H). MS: 420 (MH)⁺.

### EXAMPLE 4

### Preparation of Compound 5′

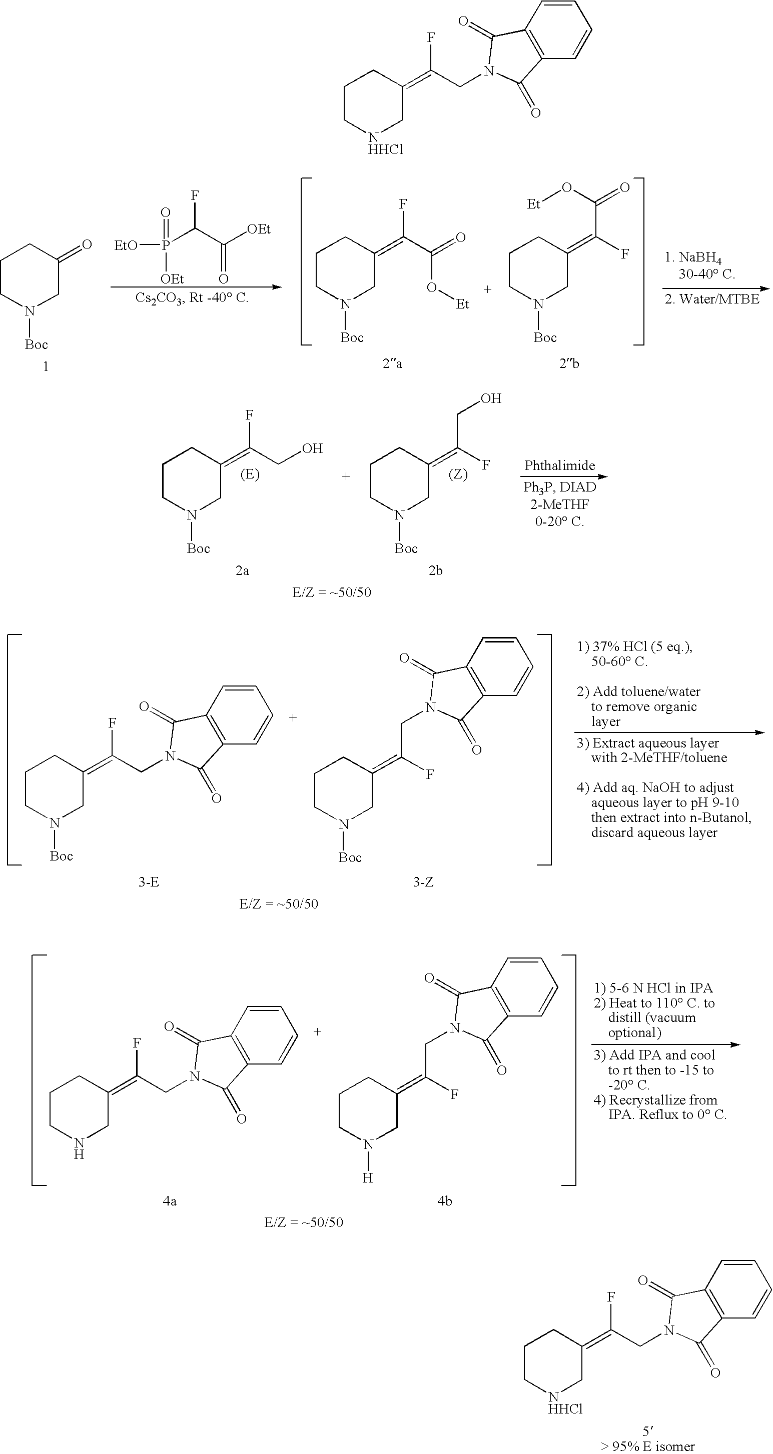

### Preparation of 3-(1-fluoro-2-hydroxyethylidene)piperidine-1-carboxylic acid tert-butyl ester (2a and 2b)

A 50-L jacketed glass reactor, equipped with a thermocouple, overhead air stirrer, two air condensers, and nitrogen inlet, was charged with N-Boc-3-piperidone (1, 2.00 kg, 10.04 mol), ethanol (22.2 L) and 2-fluorotriethylphosphonoacetate (2.54 kg, 10.50 mol). The mixture was stirred to obtain a homogeneous solution and then Cs₂CO₃ was added in portions over 10 minutes. After the Cs₂CO₃ addition was complete, reaction completion affording a ˜50:50 mixture of 2″a and 2″b was determined by HPLC. Next, NaBH₄ was added in portions over 3-4 h; during most of this addition the reaction temperature was maintained between 40° C. to 55° C.

Additional EtOH (8.0 L) was added to maintain stirring of the thickening suspension. The reaction was allowed to stir overnight, after which time HPLC analysis indicated that the reaction was complete. The reaction mixture was transferred to a stirred 100-L glass-lined reactor containing water (50.0 L). The aqueous mixture was extracted with methyl t-butyl ether (25.0 L). Concentration afforded 2a and 2b (2.60 kg, 106%) of as a 50:50 (E:7) mixture (HPLC).

### Preparation of 2-[2-fluoro-2-(3-piperidinylidene)ethyl]-1H-Isoindole-1,3)-dione hydrochloride (5′)

A 100-L Hastalloy® reactor was charged with 2a and 2b as ˜50:50 (E:Z) mixture (4.90 kg, 19.98 mol) dissolved in 2-MeTHF (39.5 L), phthalimide (3.4 kg, 23.17 mol) and Ph₃P (6.4 kg, 24.37 mol). The white suspension was stirred under N₂ and cooled to 0-5° C. DIAD (4.3 kg, 20.18 mol) was added via a metering pump over 0.5 h, while the reaction temperature was maintained at <25° C. After the addition, the reaction was stirred at 20-25° C. for 2 h to achieve reaction completion (HPLC). Upon completion, concentrated hydrochloric acid (9.8 kg) was added and the reaction mixture was heated to 50-60° C. for 1 h then cooled to 20-25° C. After confirming reaction completion (HPLC) to 3-E and 3-Z, water (19.7 L) and toluene (34.1 L) were added to the stirring mixture. After settling, the organic phase was discarded and the aqueous phase (pH≦1) was washed with 2-MeTHF (19.7 L) and toluene (19.7 L). The aqueous phase was cooled to 5-10° C. and the pH was adjusted to 10-11 by adding 50% aq. NaOH (5.7 kg) via a metering pump, while the reaction temperature was maintained at <15° C. The aqueous phase was extracted twice with n-butanol (39.5 L and 14.8 L). To the combined organic phase of 4a and 4b, 5-6N HCl in 2-propanol

(6.0 kg) was added adjusting the pH to 0-1. Distilled (atmospheric, then vacuum) off most of the n-butanol to ˜15 L of volume and cooled 50-70° C. To the concentrated n-butanol solution were added 2-propanol (76.4 L) and 5-6N HCl in 2-propanol (0.9 kg). The product precipitated upon cooling to room temperature. After stirring overnight, the slurry was cooled to −15 to −20° C. and the product was isolated via filtration. The wet filter cake was dried (60 Torr, 65° C.) to a constant weight to give 2.085 kg (29% mass yield) of crude 5′ (HPLC showed E:Z ratio of 72:28). Recrystallization in 2-propanol, heated to reflux and cooled to 0-5° C. affords >95% desired E-isomer 5′ in 18-22% overall yield.

### EXAMPLE 5

### Preparation of Compound 12

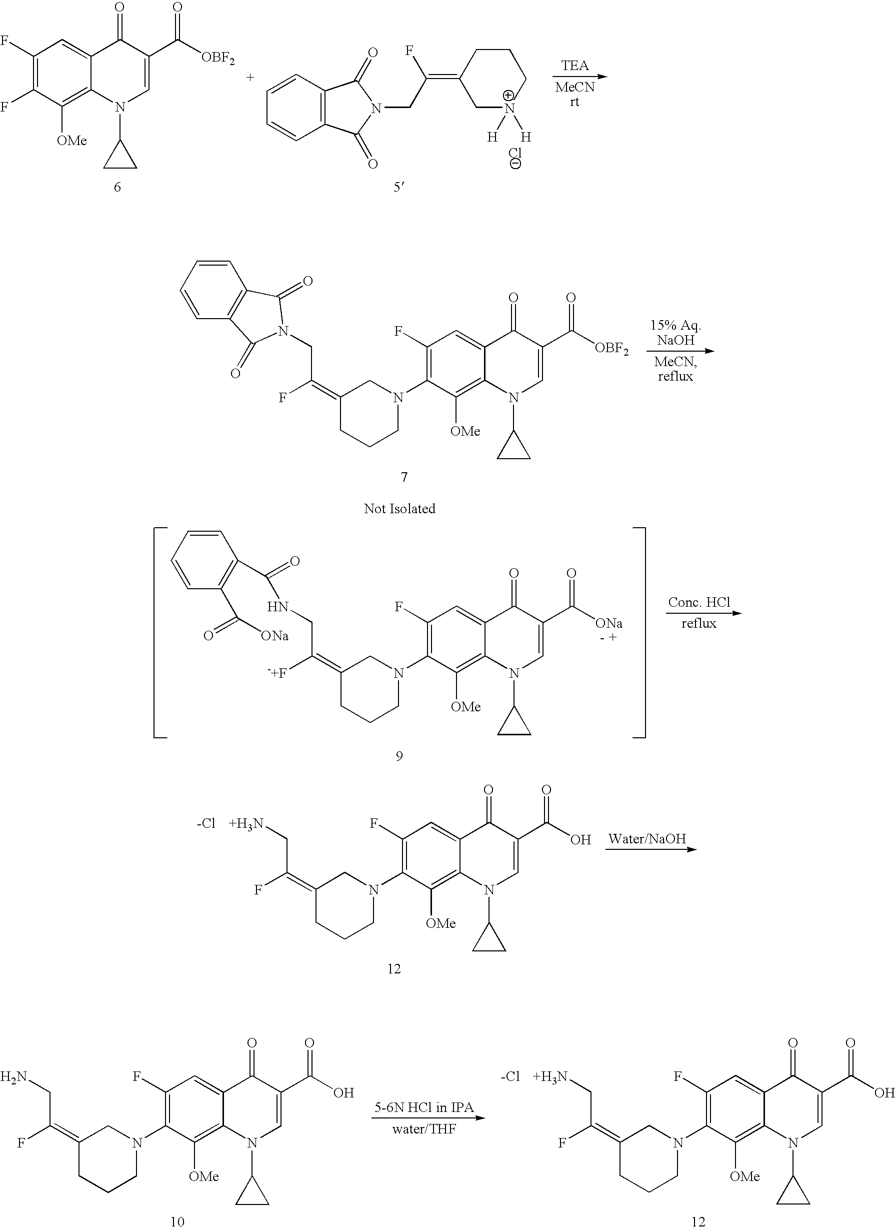

### Preparation of intermediate 7

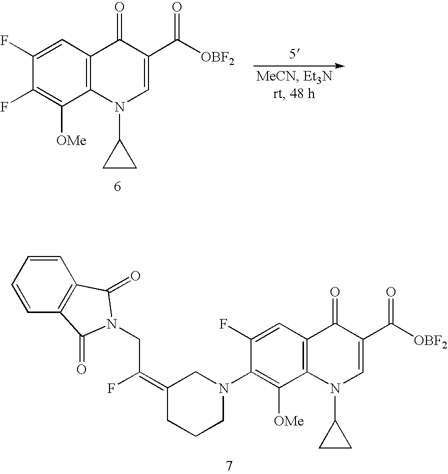

A 100-L Hastalloy® reactor was charged with difluoroborate ester 6 (2.86 kg, 91.5 HPLC wt % 6.92 mol), MeCN (29.0 L) and piperidine 5′ (2.40 kg, 82.7 HPLC wt % 6.42 mol). This suspension was stirred at 20° C. under N₂ for 10 min, Et₃N (300 mL, 2.147 mol) was added to the stirred mixture over an additional 5-min period. After this addition, the mixture was stirred for a minimum of 48 h, to achieve reaction completion. The product, 7, was isolated via filtration and washed well with water (23 L) followed by a 1:1 mixture of water:MeCN (11.4 L). After vacuum drying (60 Torr, 80° C.) the wet filter cake to a constant weight, obtained 7 (2.87 kg, 75% yield) as a yellow solid. HPLC analysis showed 88% by weight and 95% by area.

### Preparation of 7-[3-(2-amino-1-fluoro-ethylidene)-piperidin-1-yl]-1-cyclopropyl-6-fluoro-8-methoxy-4-oxo-1,4-dihydro-quinoline-3-carboxylic acid (10)

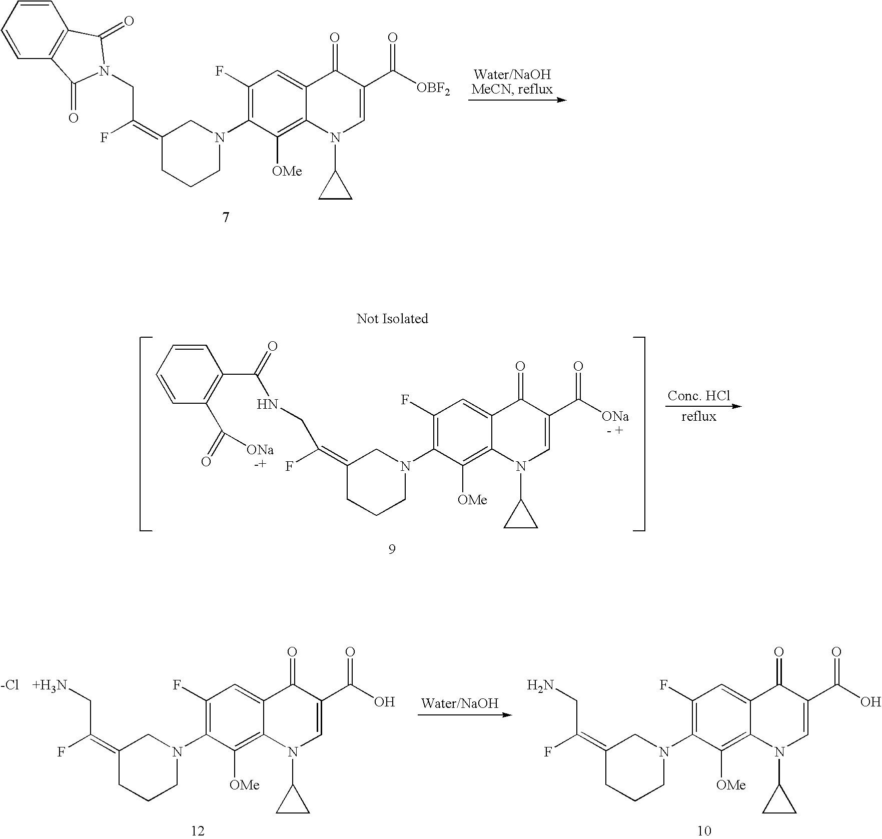

A 500-mL 3-neck round bottom flask equipped with an overhead stirrer, thermocouple, condenser, pressure-equalizing addition funnel and a nitrogen inlet adapter was charged with 7 (10.4 g, 88 HPLC wt %, 15.3 mmol), MeCN (40 mL) and 15% aq. NaOH (47.2 g, 174 mmol). The resultant slurry was heated to reflux (78-82° C.) for 2 h. Then conc. HCl (20 mL, 240 mmol) was added and continued refluxing for another 2 h. Upon cooling to room temperature, the resultant slurry was filtered and washed with THF (34 mL) to afford 7.05 g of crude 12 (contains ˜8-9% of 8 as an impurity). Added 6.8 g of crude 12 to water (150 mL) and heated to 85-95° C. for 1 h. Cooled to ˜30-40 C and filtered off undissolved impurity 8 and washed with hot water (2×10 mL). To the clear yellow filtrate added 6N sodium hydroxide (1.8 mL, ˜11 mmol) to adjust the pH to ˜6 and precipitate 12. Filtered the slurry, washed with water (10 mL) and dried under vacuum (60-65° C., 27-28″ Hg) to afford 10 (4.61 g, 74% yield) as a yellow solid. HPLC analysis showed 99.3% by area.

### Preparation of 7-[3-(2-Amino-1-fluoro-ethylidene)-piperidin-1-yl]-1-cyclopropyl-6-fluoro-8-methoxy-4-oxo-1,4-dihydro-quinoline-3-carboxylic acid hydrogen chloride salt (12)

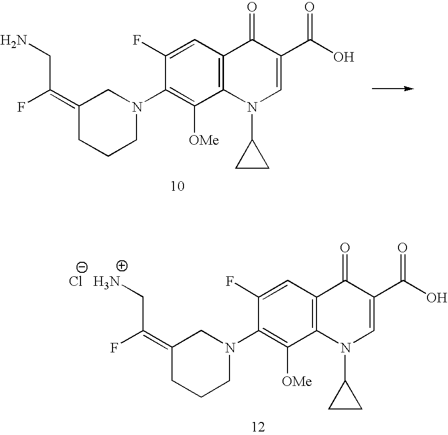

A 250 mL 3-necked round bottom flask equipped with an overhead stirrer, thermocouple, condenser, pressure-equalizing addition funnel, and a nitrogen inlet adapter was charged with IPA (30 mL), compound 10 (6.0 g, 14.3 mmol), 5/6N HCl in IPA (2.5 mL, 15.0 mmol) and water (9.0 mL). The suspension was stirred under N₂ and heated to ˜75° C. After cooling to 70° C., filtered the solution and transferred the filtrate to a clean 250 mL 3-necked round bottom flask. Solids precipitated upon cooling to room temperature. Diluted the slurry with THF (96 mL) and cooled to 0-5° C. with stirring. Filtered the slurry, washed with THF (20 mL) and air dried to afford 12 (5.18 g, 76.4% yield) as an off-white to very light-yellow solid.

HPLC analysis showed 100% by area. Elemental analysis: % C 53.39, % H 5.29, % N 8.85, % Cl 7.49, % F 7.91. KF=3.45%

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A method for making one or more compounds of Formula (1), 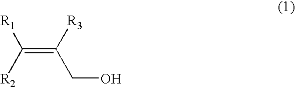
wherein
R¹ and R² are independently selected from H, aryl, C₁₋₁₀alkyl, C₂₋₁₀alkenyl, and C₃₋₁₀alkynyl, or R¹, R² and the C atom they attach to may together form C₃₋₁₀cycloalkyl or heterocyclyl; and
R³ is H, C₁₋₁₀alkyl, C₂₋₁₀alkenyl, C₃₋₁₀alkynyl, halogen, aryl, heteroaryl, or heterocyclyl,
said method comprising
(a) reacting, in the presence of one or more bases, one or more compounds of Formula (i) 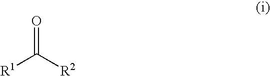
wherein R¹ and R² are independently selected from H, aryl, C₁₋₁₀alkyl, C₂₋₁₀alkenyl, and C₃₋₁₀alkynyl, or R¹, R² and the C atom they attach to may together form C₃₋₁₀cycloalkyl or heterocyclyl, with a compound of Formula (ii) 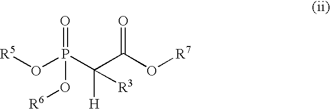
wherein R³ is selected from H, C₁₋₁₀alkyl, C₂₋₁₀alkenyl, C₃₋₁₀alkynyl, halogen, aryl, heteroaryl, and heterocyclyl, and R⁵, R⁶, and R⁷ are independently selected from C₁₋₁₀alkyl and aryl; and
(b) adding one or more reducing agents into the reaction of step (a).

2. The method of claim 1 wherein the compound of Formula (1) is 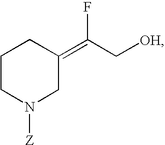
wherein Z is selected from —C(O)O—C(CH₃)₃, —C(O)OCH₂Ph, —C(O)-Ph, —C(O)CH₃, —S(O)₂-PhCH₃, and —S(O)₂—CH₃.

3. The method of claim 1 wherein the compound of Formula (1) is

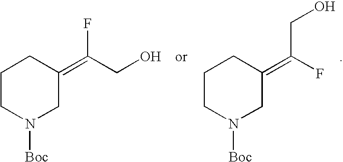

4. The method of claim 1 wherein the compound of Formula (1) consists of

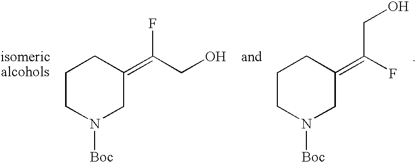

5. The method of claim 1 wherein the compound of Formula (i) is in one or more solvents independently selected from alcohols, 2-methoxyethanol, diols, polyols, polyethers, polyethylene glycol monomethyl ether derivatives, DMA, DMF, pyridine, and Et₃N.

6. The method of claim 5 wherein the solvent is one or more alcohols, each alcohol having 1-6 carbon atoms.

7. The method of claim 6 wherein the solvent is 2-methoxyethanol or ethanol.

8. The method of claim 1, 5, 6, or 7 wherein the compound of Formula (i) is

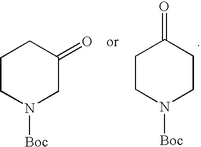

9. The method of claim 1 wherein the compound of Formula (i) is in one or more solvents independently selected from THF, Et₂O, and toluene.

10. The method of claim 1 wherein the base is at least one member selected from metal carbonates, bicarbonates, metal hydroxides, and organic bases.

11. The method of claim 10 wherein the base is at least one member selected from Cs₂CO₃, K₂CO₃, KOt-Bu, Li₂CO₃, Na₂CO₃, LiOH, NaOH, KOH, Et₃N, DBU, DABCO, and pyridine.

12. The method of claim 11 wherein the base is Cs₂CO₃.

13. The method of claim 1 wherein the reducing agent is one or more metal borohydrides.

14. The method of claim 13 wherein the reducing agent is at least one member selected from NaBH₄, LiBH₄, KBH₄, Ca(BH₄)₂, and Zn(BH₄)₂.

15. The method of claim 13 further comprising adding a compatible salt in step (b).

16. The method of claim 14 wherein the reducing agent is NaBH₄ and the compatible salt is LiCl or CaCl₂.

17. The method of claim 1 wherein the compound of Formula (i) is in polyethers, Et₃N, THF, Et₂O, or toluene, and the reducing agent is at least one member selected from DIBAL and LAH.

18. The method of claim 1 wherein
the compound of Formula (1) is 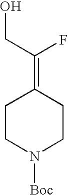
or a mixture of 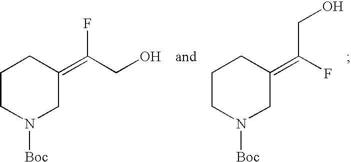
the compound of Formula (i) is selected from 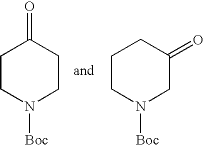
, said
compound of Formula (i) is in the solvent of 2-methoxyethanol;
the base is Cs₂CO₃; and
the reducing agent is NaBH₄.

19. The method of claim 1 wherein 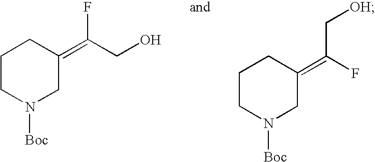
the compounds of Formula (1) are 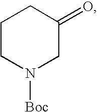
the compound of Formula (i) is said compound of Formula (i) is in the solvent of 2-methoxyethanol;
the base is Cs₂CO₃; and
the reducing agent is NaBH₄.

20. The method of claim 1 comprising 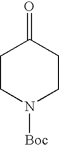
(a) reacting in the solvent of 2-methoxyethanol
with Cs₂CO₃ and 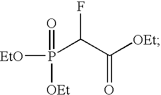
and 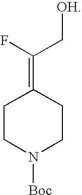
(b) adding NaBH₄ into the reaction of step (a) to form

21. The method of claim 1 comprising 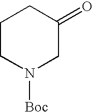
(a) reacting in the solvent of 2-methoxyethanol with Cs₂CO₃ and 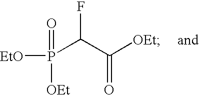
(b) adding NaBH₄ into the reaction of step (a) to form a mixture of

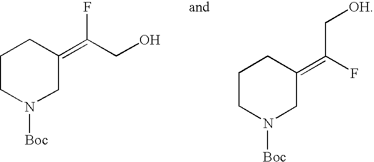

22. The method of any of claims 1-21 wherein both steps (a) and (b) are done in one reaction vessel.

23. The method of claim 1 further comprising
(c) a liquid-liquid extraction with a two-phase mixture composed of a polar and a non-polar phase after step (b).

24. A method for making 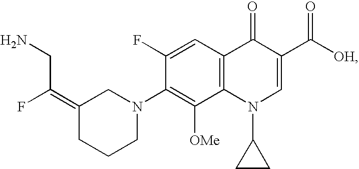
said method comprising 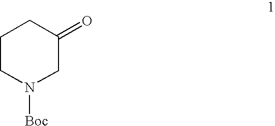
(a) reacting in the presence of one or more bases 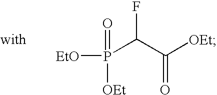
(b) adding one or more reducing agents into the reaction of step (a) to form 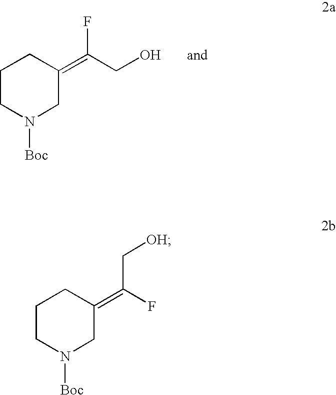
(c) extracting 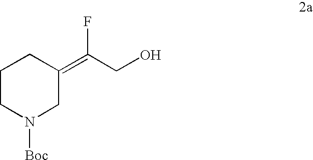
with hexane or heptane;
(d) converting 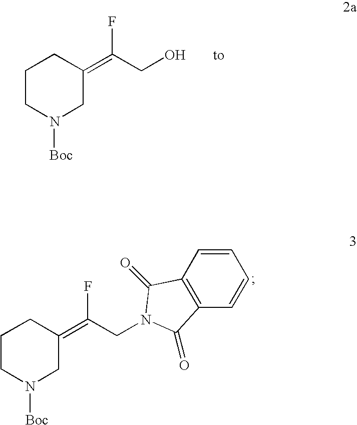 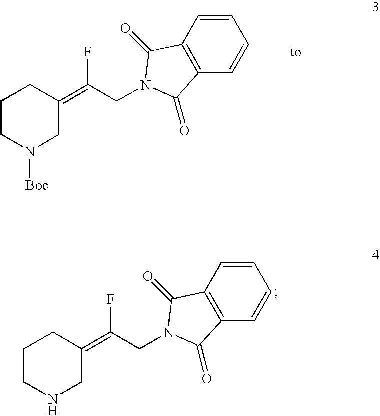
(e) converting 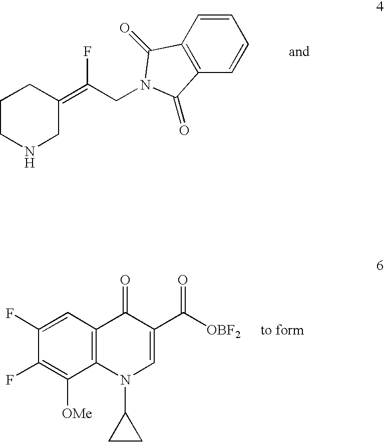
(f) reacting 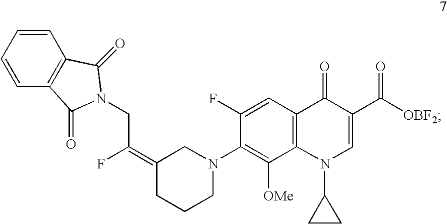 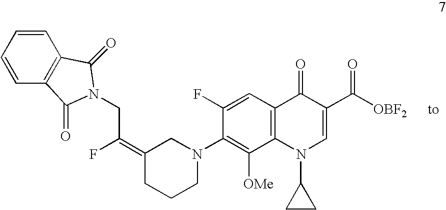
(g) converting 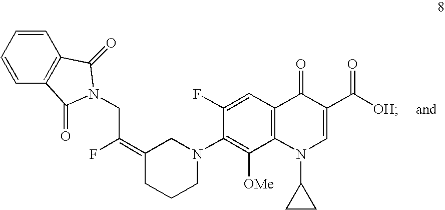 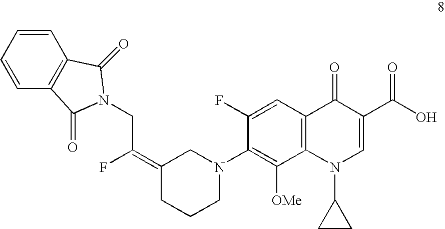
(h) adding H₂NNH₂ into and MeOH.

25. The method of claim 24 further comprising conversion of 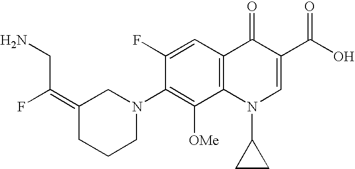
to 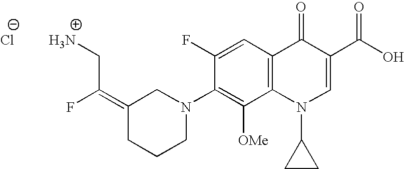
with HCl.

26. The method of claim 24 further comprising conversion of 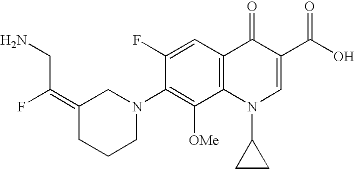
to 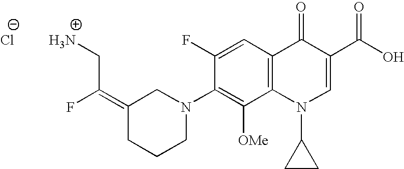
in EtOH and HCl.

27. The method of claim 24 further comprising a step of recrystallizing 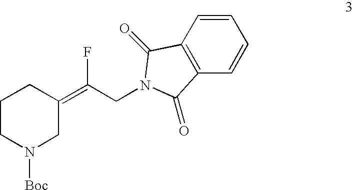
between step (d) and step (e).

28. A method for making
said method comprising 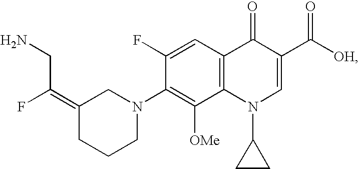
(a) reacting 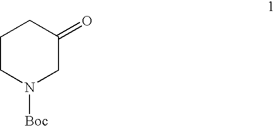
in the presence of one or more bases 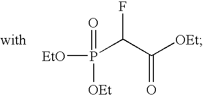
(b) adding one or more reducing agents into the reaction of step (a) to form 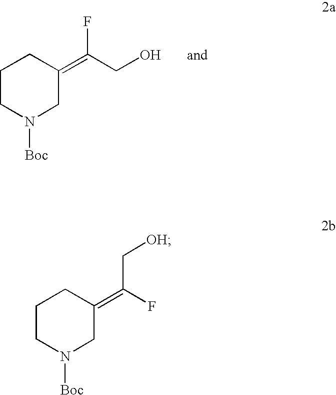
(c) extracting 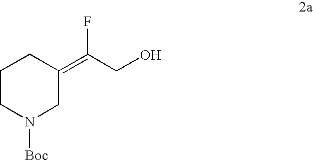
with hexane or heptane; 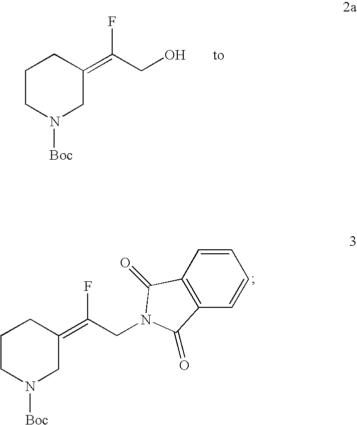
(d) converting 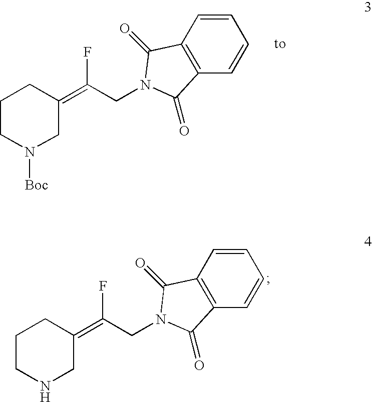
(e) converting 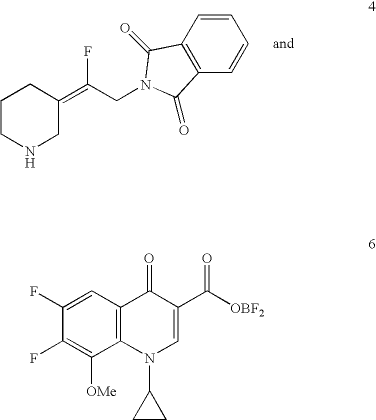
(f) reacting to form 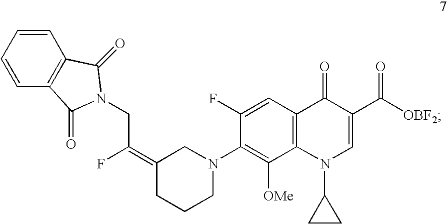 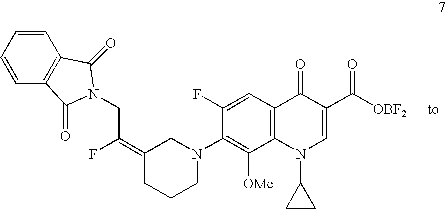
(g) converting 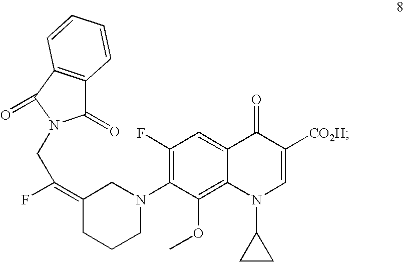 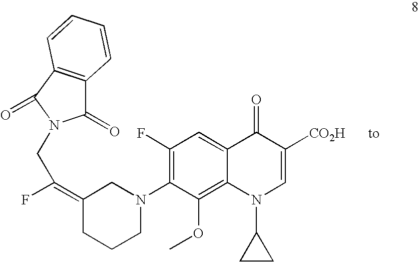
(h) converting 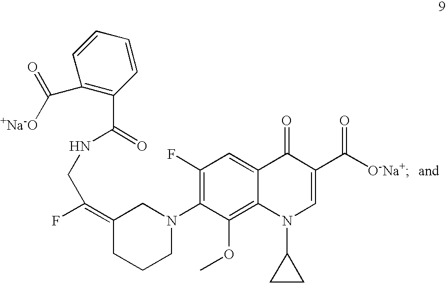
(i) adding MeOH and H₂SO₄, sequentially, into the reaction of step (h).

29. A method for separating isomeric alcohols of Formula (1) in an aqueous mixture 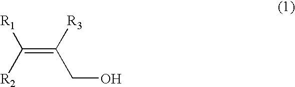
wherein
R¹ and R² are different groups selected from H, C₁₋₁₀alkyl, C₂₋₁₀alkenyl, and C₃₋₁₀alkynyl, or R¹, R² and the C atom they attach to may together form an asymmetric group selected from substituted C₃₋₁₀cycloalkyl and optionally substituted heterocyclyl;
R³ is H, unsubstituted C₁₋₁₀alkyl, halogen, aryl, or heterocyclyl, said method comprising
(a) contacting an aqueous mixture of the isomeric alcohols with an adequate volume of a non-polar solvent; and
(b) separating the resulting non-polar solvent from the aqueous layer.

30. The method of claim 29 wherein R¹, R² and the C atom they attach to together form an asymmetric group selected from 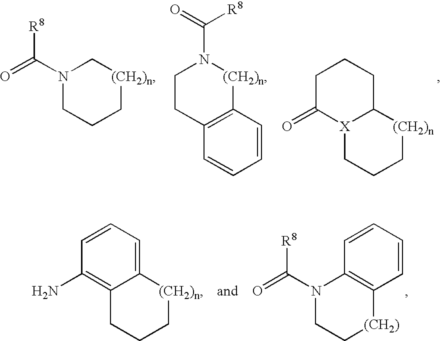
wherein
n is 0-4;
X is N or CH; and
R⁸ is C₁₋₁₀alkyl, C₁₋₁₀alkoxy, aryloxy, or aryl,
provided that the C atom R¹ and R² attach to is not next to a N atom in the asymmetric group.

31. The method of claim 30 wherein the asymmetric group is selected from

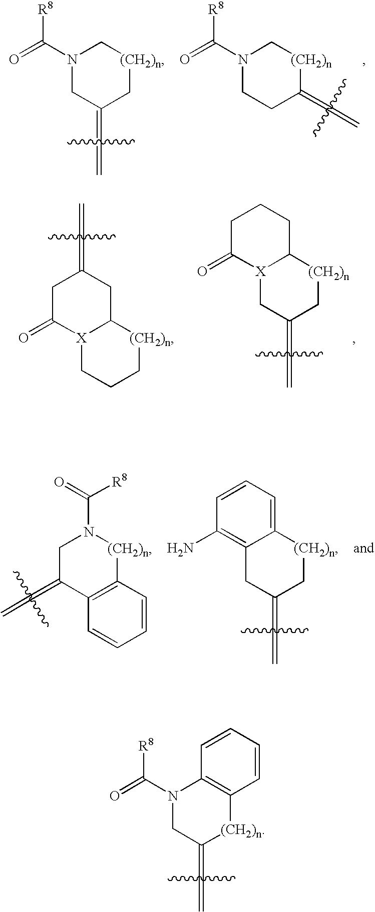

32. The method of claim 30 wherein the asymmetric group is selected from

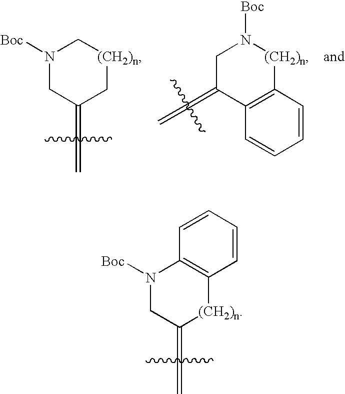

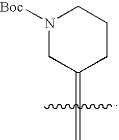

33. The method of claim 30 wherein the asymmetric group is

34. The method of claim 29, 30, 31, or 32, further comprising
(c) contacting the aqueous layer with an adequate volume of a water-insoluble polar solvent.

35. The method of claim 34 wherein the water-insoluble polar solvent is methyl tert-butyl ether or ethyl acetate.

36. The method of claim 34 wherein the non-polar solvent is hexane or heptane.

37. The method of claim 34 wherein the non-polar solvent is hexane or heptane and the polar solvent is methyl tert-butyl ether.

38. The method of claim 29, 30, 31, or 32 wherein the non-polar solvent is hexane or heptane.

39. A method for separating isomers of Formula (2) in an n-butanol solution 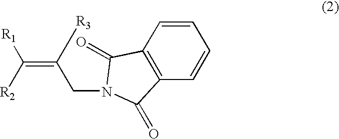
wherein
R¹, R² and the C atom they attach to together form 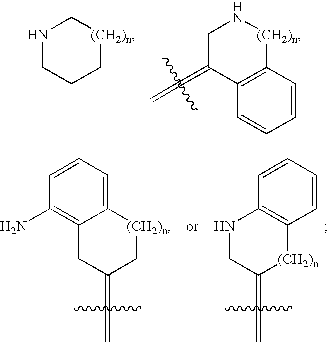
R³ is H, unsubstituted C₁₋₁₀alkyl, halogen, aryl, or heterocyclyl; and
n is 0-4,
said method comprising
(a) contacting an aqueous mixture of the isomers of Formula (2) with an adequate volume of a mixture of HCl and IPA;
(b) heating the resulting solution to a temperature from about 85° C. to about 118° C.; and
(c) adding IPA into the resulting solution.

40. The method of claim 39 wherein the mixture of HCl and IPA is 5-6N HCl in 2-propanol.

41. The method of claim 39 wherein vacuum is applied in step (b).

42. The method of claim 39 wherein the solution in step (b) is heated to about 110° C.

43. The method of claim 39, further comprising
(d) cooling the resulting solution to a temperature between r.t. and −20° C.

44. The method of claim 43 wherein the temperature in step (d) is between −15 and −20° C.

45. The method of claim 39 wherein
isomers of Formula (2) are 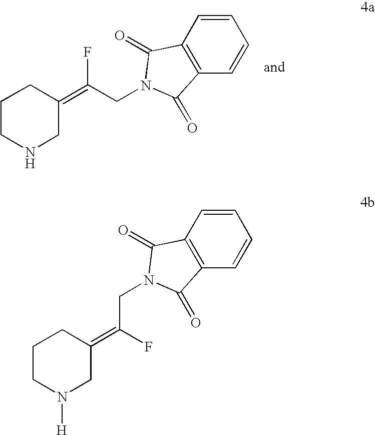
in n-butanol;
the mixture of HCl and IPA is 5-6N HCl in 2-propanol; and
the solution in step (b) is heated to about 110° C. under vacuum.

46. A method for making 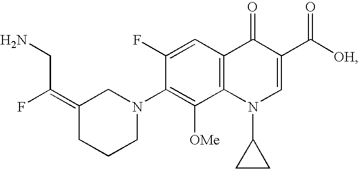
said method comprising 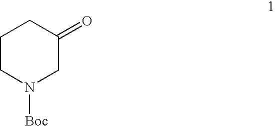
(a) reacting in the presence of one or more bases 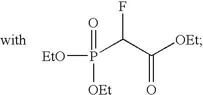
(b) adding one or more reducing agents into the reaction of step (a) to form 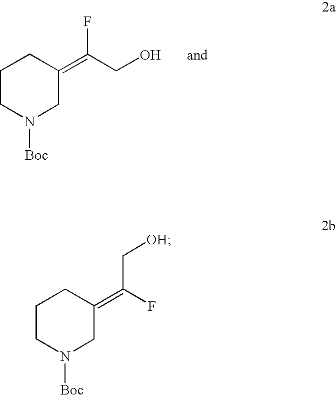 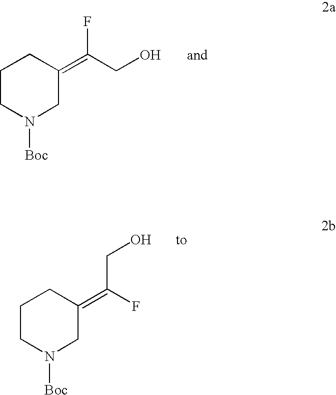
(c) converting 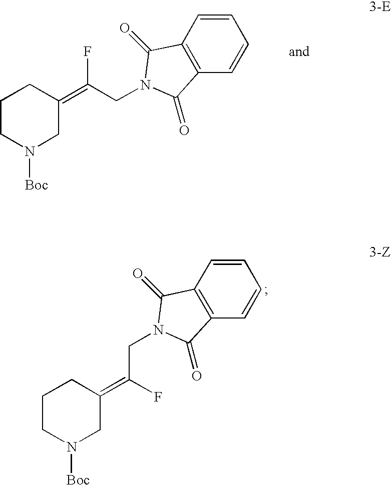 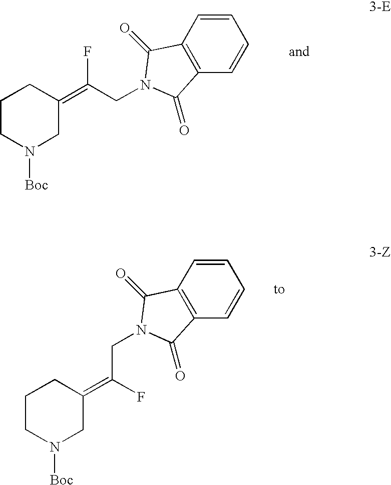
(d) converting 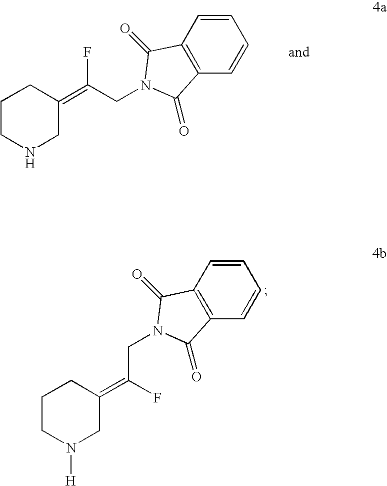
(e) adding 5-6 N HCl in IPA into the reaction of step (d);
(f) heating the reaction of step (e) to about 110° C.; 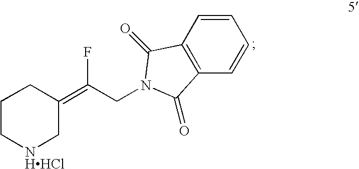
(g) adding IPA to precipitate 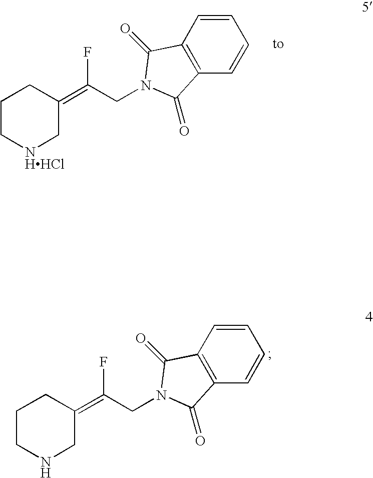
(h) converting 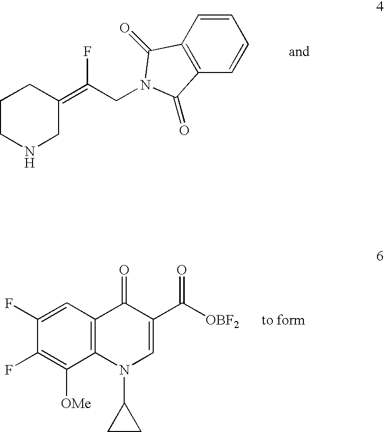
(i) reacting 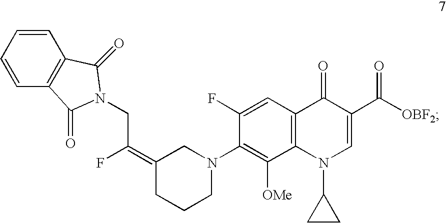
(j) converting 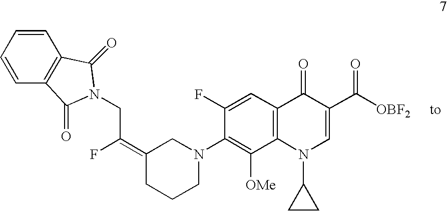 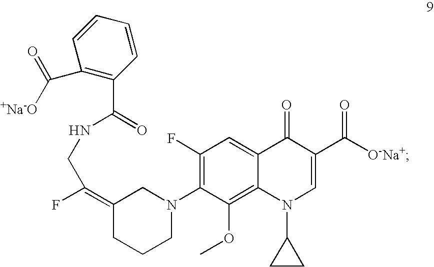 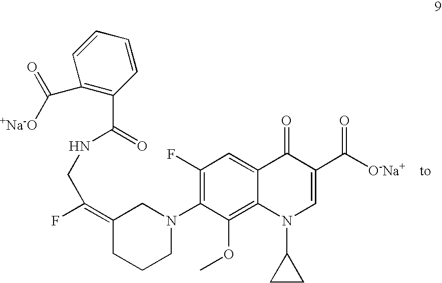
(k) converting 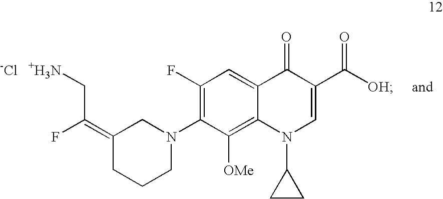
(l) converting

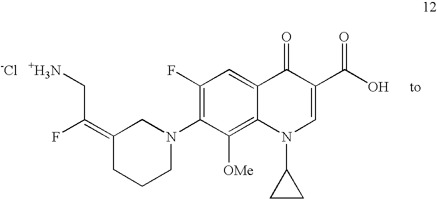

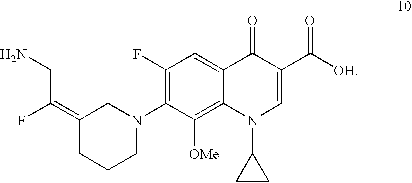

47. A method for making 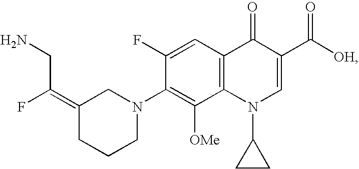
said method comprising 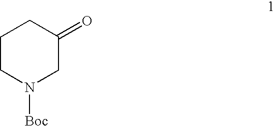
(a) reacting in the presence of one or more bases 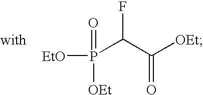
(b) adding one or more reducing agents into the reaction of step (a) to form 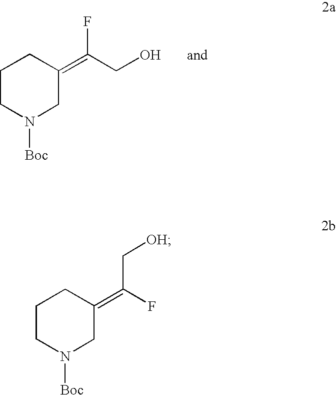 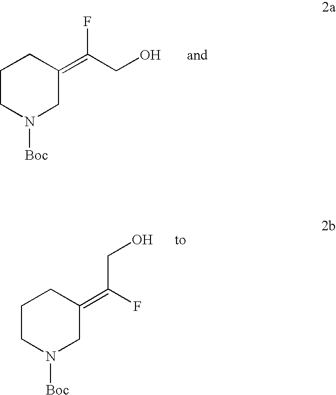
(c) converting 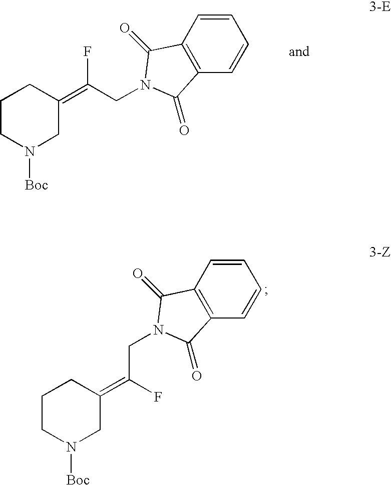 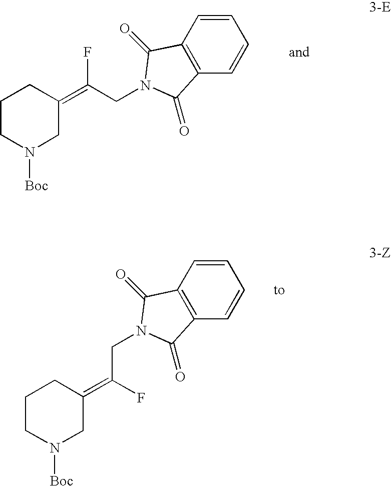
(d) converting 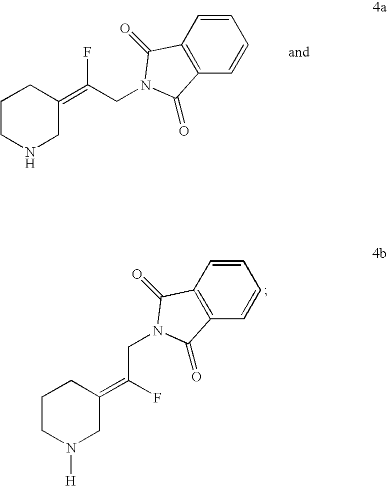
(e) adding 5-6 N HCl in IPA into the reaction of step (d);
(f) heating the reaction of step (e) to about 110° C.; 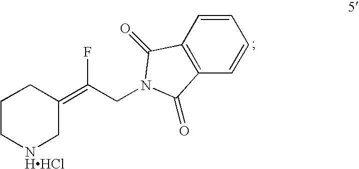
(g) adding IPA to precipitate 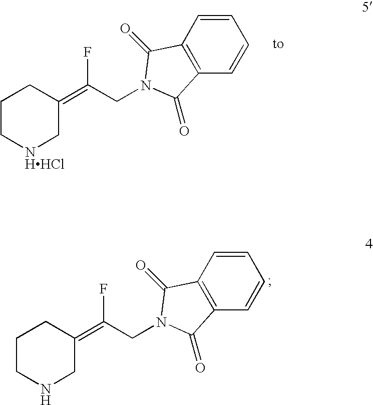
(h) converting 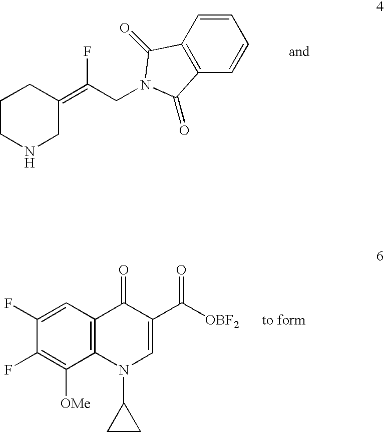
(i) reacting 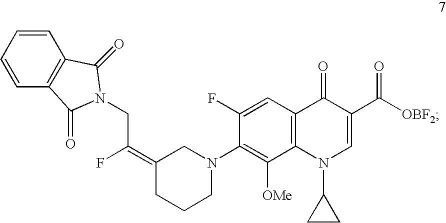 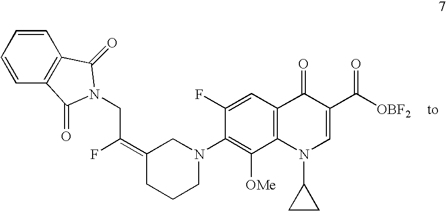
(j) converting 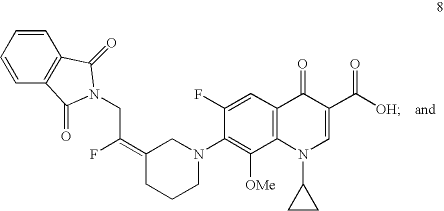 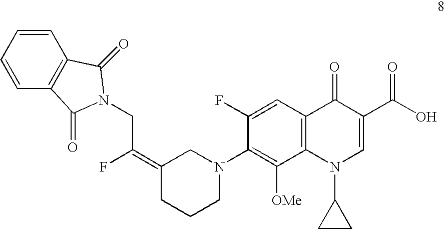
(k) adding MeOH into and H₂NNH₂.

48. The method of claim 46 or 47 wherein one or more extractions using one or more solvents selected from alcohol and non-polar aprotic is performed in step (d).

49. The method of claim 48 wherein the solvent is selected from 2-propanol, 2-MeTHF, toluene, diethyl ether, ethyl acetate, MTBE, and n-butanol.

50. The method of claim 49 wherein the solvents are 2-MeTHF and toluene.

51. The method of claim 49 wherein the solvent is n-butanol.

52. The method of claim 46 or 47 wherein one extraction with 2-MeTHF and toluene is performed followed by another extraction with n-butanol.
